(19) United States
(12) Patent Application Publication
(10) Pub. No.: US 2025/0082627 A1
(43) Pub. Date: Mar. 13, 2025

(54) TOBACCO HARM REDUCTION AND PAIN RELIEF PRODUCTS AND METHODS THEREOF

(71) Applicant: Ready Mix Naturals, LLC, Seguin, TX (US)

(72) Inventors: Charlotte L. OWEN, Seguin, TX (US); Richard AVILA, JR., Prescott Valley, AZ (US); Geoff W. HABICHT, Phoenix, AZ (US)

(73) Assignee: Ready Mix Naturals, LLC, Seguin, TX (US)

(21) Appl. No.: 18/826,821

(22) Filed: Sep. 6, 2024

Related U.S. Application Data
(60) Provisional application No. 63/581,784, filed on Sep. 11, 2023.
(60) Provisional application No. 63/642,241, filed on May 3, 2024.

(51) Int. Cl. A61K 31/465; A24B 15/167; A24F 47/00; A61K 31/496; A61K 31/5377

(52) U.S. Cl. CPC A61K 31/465; A24B 15/167; A24F 47/00; A61K 31/496; A61K 31/5377

(57) ABSTRACT

Tobacco harm reduction (THR) products according to the present disclosure include both dermally and orally administered products, each comprising at least one substituted pyridine compound according to Formula (I), with excipients and optional substrate customized to a physical form or a function of the product. In various examples, THR products include (a) a transdermal patch, (b) a dissolving film, (c) a lozenge, (d) a chewing gum, (e) an oral pouch, (f) impregnated toothpicks, (g) impregnated or coated dental floss, (h) a mouthwash, (i) a gummy candy, (j) sublingual disintegrating tablets, or (k) a topical/transdermal cream.

### CROSS-REFERENCE TO RELATED APPLICATIONS

This application claims the benefit of and priority to U.S. Provisional Patent Application Ser. No. 63/581,784 filed Sep. 11, 2023 and entitled “Tobacco Harm Reduction And Pain Relief Products And Methods Thereof” and U.S. Provisional Patent Application Ser. No. 63/642,241 filed May 3, 2024 entitled “Tobacco Harm Reduction And Pain Relief Products And Methods Thereof”. These disclosures are incorporated herein by reference in their entireties for all purposes.

### FIELD

The present disclosure generally relates to products and methods for the cessation of tobacco smoking and elimination of the associated harm from smoking, and in particular to nicotine-free oral and dermal tobacco harm reduction products capable of promoting smoking cessation when used alone or in combination with products for relieving pain.

### BACKGROUND

It is common knowledge that smokers can experience health benefits from cigarette cessation, and in general, most adult smokers in the U.S. express a desire to quit smoking. Electronic Nicotine Delivery Systems (ENDS), depending on the composition of the vaporizable liquid, should, at least in principle, provide a viable cessation aid to smoking. However, various studies have suggested that users of electronic devices to self-administer nicotine are actually not more likely to quit smoking tobacco products. See, for example, B. Kaplan, et al., “Effectiveness of ENDS, NRT and Medication for Smoking Cessation Among Cigarette-Only Users,” Tob Control 2023; 32:302-307 and R. El Dib, et al., “Electronic Nicotine Delivery Systems and/or Electronic Non-Nicotine Delivery Systems for Tobacco Smoking Cessation or Reduction: A Systematic Review and Meta-Analysis,” BMJ Open 2017; 7:e012680.

Cessation of tobacco smoking requires commitment and compliance with alternative products. In the case of a smoker, compliance with alternative products relies on having identical or at least close to identical sensory effects as would be experienced from inhaling tobacco smoke from a burning cigarette. To date and evidenced by the data showing a lack of cessation, this has not been accomplished in the industry.

Therefore, new alternatives to smoking are needed that can provide an acceptable personal experience to a previous tobacco smoker. In particular, non-nicotine products are needed for use singly or in combination to reduce the personal harm from tobacco.

### SUMMARY

It has now been surprisingly discovered that non-nicotine tobacco harm reduction products, optimized to provide a balance between senses when used orally and/or dermally, promote smoking cessation. In certain instances, a combination of topical non-nicotine pain relief and oral or transdermal administration of non-nicotine alkaloids capable of stimulating nicotinic acetylcholine receptors (nAChRs), act synergistically to promote nicotine cessation.

In various embodiments of the present disclosure, tobacco harm reduction (THR) products include both dermally and orally administered products, each comprising at least one substituted pyridine compound and excipients customized to the physical form of the product.

In various examples, THR products include, but are not limited to, dissolving film strips, lozenges, chewing gum, transdermal patches, oral pouches, impregnated toothpicks, impregnated dental floss, mouthwash, gummy candies, sublingual disintegrating tablets, topical creams, pain relief patches, an oral spray, inhaler, nasal spray, alkaloid dispensing pouch, e-cigarette, electronic nicotine delivery system, or comparable nicotine replacement product.

In certain examples, a smoking cessation program comprises use of one or a combination of two or more of THR products. Disclosed is a smokeless THR product. In some embodiments, the smokeless THR product is adapted to be chewed, sucked, applied, or orally manipulated in a consumer's mouth. In some embodiments, wherein the active ingredient of the the smokeless THR product is about 0.1% to about 10% by weight based on the weight of the the smokeless THR product.

In certain embodiments, a smoking cessation program comprises use of an oral or dermal THR product in combination with a topical or transdermal pain relief product. These methods ensure that an individual desirous of tobacco cessation has a product capable of stimulating nicotinic acetylcholine receptors (nAChRs) and a product to alleviate perceived pain from not smoking.

In various embodiments, a tobacco harm reduction (THR) product comprises:
at least one substituted pyridine compound of Formula (I);

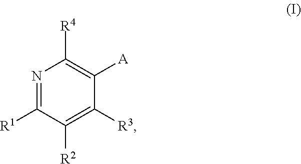

or a salt, or mixed salt thereof, wherein:
A is selected from:

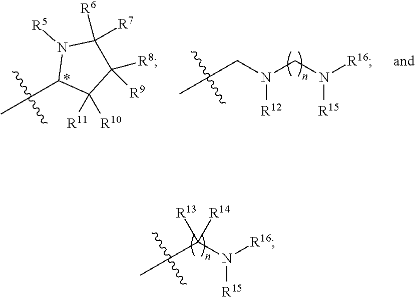

R¹, R², R³, R⁴ and R⁵ are independently selected from H, alkyl, substituted alkyl, cycloalkyl, substituted cycloalkyl, alkenyl, substituted alkenyl, and

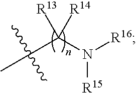

R⁶ and R⁷ are independently selected from H, alkyl, substituted alkyl, cycloalkyl, substituted cycloalkyl, alkenyl and substituted alkenyl, or R⁶ and R⁷ together are =O;
R⁸, R⁹, R¹⁰, R¹¹, R¹², R¹³, and R¹⁴ are independently selected from H, alkyl, substituted alkyl, cycloalkyl, substituted cycloalkyl, alkenyl, and substituted alkenyl;
n is an integer from 1 to 10; and
R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered optionally substituted heterocyclic ring,
with the proviso that if A is

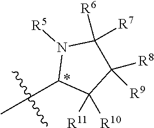

and R⁵ is methyl, then R¹, R², R³, R⁴, R⁶, R⁷, R⁸, R⁹, R¹⁰, and R¹¹ cannot all be H; and
optionally, a substrate,
wherein the THR product is entirely devoid of (R) or (S) nicotine.

In various embodiments, the THR product further comprises at least one excipient, optionally, at least one organic acid, optionally, at least one chemosensory irritant and combinations thereof.

In various embodiments, the excipient is selected from binders, diluents, syrups, sugars, sweeteners, solubilizers, solvents, polyols, emollients, humectants, oils, polymers, surfactants, carriers, stabilizers, and preservatives. The excipient and the optional substrate contribute to a physical form or a function of a THR product.

In various embodiments, the substrate is selected from nonwoven fabrics, latex, rubber or fabric swatches, wood splints, and a string or tape, such that the THR product form is an oral alkaloid dispending pouch, a transdermal or topical THR or pain relief patch, an impregnated toothpick, or impregnated/coated dental floss or dental tape, respectively.

### DETAILED DESCRIPTION

The detailed description of exemplary embodiments makes reference to the accompanying drawings, which show exemplary embodiments by way of illustration and their best mode. While these exemplary embodiments are described in sufficient detail to enable those skilled in the art to practice the invention, it should be understood that other embodiments may be realized and that logical, chemical, and mechanical changes may be made without departing from the spirit and scope of the inventions. Thus, the detailed description is presented for purposes of illustration only and not of limitation. For example, unless otherwise noted, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

In various embodiments of the present disclosure, products and methods for tobacco harm reduction (THR) and associated pain relief as part of THR are disclosed, wherein each of the product forms comprise at least one substituted pyridine compound, and wherein each product is entirely absent (R) or (S) nicotine. In various examples, product forms include, but are not limited to, dissolving films, lozenges, chewing gum, transdermal patches, oral pouches, impregnated toothpicks, impregnated dental floss, mouthwash, gummy and other candies, sublingual disintegrating tablets, topical creams, and pain relief patches. In various examples, THR methods comprise simultaneous or sequential use of a combination of THR products.

### Definitions and Interpretations

Unless defined otherwise, all technical and scientific terms used herein have the same meaning as commonly understood by those of ordinary skill in the chemical arts and medicinal chemistry arts to which this disclosure relates. Although any methods and materials similar or equivalent to those described herein can be used in the practice or testing of the subject matter of the present disclosure, preferred methods and materials are described. As per common practice in organic chemistry, chemical structures having a chiral center either illustrated with substituents attached by wavey bonds or not illustrated three dimensionally with wedged or dashed bonds, or not labeled (R) or (S) adjacent to the chiral center or in the corresponding structural name, is assumed to represent both enantiomers. Effort is made throughout the disclosure to mark chiral centers with an asterisk (*) at least to remind readers that the carbon marked with (*) is a chiral center and that it is possible to have (R) or (S) configuration at the (*) center. Chemical structures having multiple chiral centers not presented three dimensionally or labeled as having any particular chirality are assumed to include all possible stereoisomers. Compounds of the present disclosure comprise any physiochemical or stereochemical form they may possibly assume, such as, for example, isomers, prodrugs, active metabolites, tautomers, stereoisomers, regioisomers, solvated forms, salts, and polymorphic forms. Amorphous forms lack a distinguishable crystal lattice and therefore lack an orderly arrangement of structural units.

As used herein, the term “tobacco harm reduction” or “THR” refers to any product (a “THR product”) or any method (a “THR method”) designed to help an individual cease tobacco smoking and thus mitigate physical harm to themselves. THR products in accordance with the present disclosure include, but are not limited to, dissolving films, lozenges, chewing gum, transdermal patches, oral pouches, impregnated toothpicks, impregnated dental floss, mouthwash, gummy candies and other candy forms, sublingual disintegrating tablets, topical pain relief creams, sprays and patches. THR methods comprise the use of one of more of these product forms by an individual desirous of smoking cessation. For purposes herein, pain relief is defined as a subcategory of THR and thus comprises both THR products and methods based on pain relief. A THR product specifically for pain relief includes, but is not limited to, a topical cream, a topical spray, and a pain relief patch. In various embodiments, these and other topical products may provide pain relief by transdermal delivery of pain relief alkaloids, even though the product may be applied topically. In various methods of THR, an individual may be using an oral THR product and a topical cream at the same time. Each THR product herein comprises an alkaloid, and each product is expressly absent nicotine.

As used herein, the term “alkaloid” takes on its ordinary meaning in organic, medicinal and natural products chemistry, meaning an organic compound containing a nitrogen atom, with no further distinction whether the compound is naturally occurring or synthetically made. Tobacco Harm Reduction (THR) products of the present disclosure comprise substituted pyridine alkaloids that may be in the form of a free base compound (unprotonated), or partially or completely protonated by acids also included in the compositions, depending on molar equivalents of acid provided, and thus at least partly or even entirely present in the form of a salt in various THR products. Salts of alkaloids include any salt derived from an inorganic or organic acid, preferably an organic acid or mixtures thereof. Examples of inorganic salts include but are not limited to, salts of hydrobromic acid, hydrochloric acid, nitric acid, phosphoric acid and sulfuric acid. Examples of organic acid salts include, for example, salts of formic acid, acetic acid, trifluoroacetic acid, aspartic acid, butanoic acid, butyric acid, 2-methylbutyric acid, 3-methylbutyric acid, benzoic acid, caprylic acid, citric acid, crotonic acid, ethylenediaminetetraacetic acid (EDTA), fumaric acid, gluconic acid, glutamic acid, glyceric acid, glycolic acid, lactic acid, lauric acid, levulinic acid, maleic acid, malic acid, malonic acid, mandelic acid, methane sulfonic acid, oxalic acid, phenylacetic acid, phthalic acid, picric acid, propionic acid, pyruvic acid, salicylic acid, stearic acid, succinic acid, tannic acid, tartaric acid, tartronic acid, valeric acid, and any combinations thereof, or any other such acid presently known or yet to be discovered or synthesized. Such alkaloid salts can be prepared by reaction of an alkaloid free base compound with a suitable acid or mixture of acids in a manner known by those skilled in the art. When mixed acids are reacted with the alkaloids disclosed herein to make alkaloid salts, no attempt is made to quantify the resulting mole percentages of each individual alkaloid salt, but rather the resulting alkaloid salts are referred to as “mixed salts.” Thus, THR products herein are disclosed as comprising a substituted pyridine compound, or a salt, or mixed salt thereof.

As used herein, the term “alkyl” refers to linear or branched monovalent saturated hydrocarbon substituents, optionally substituted with one or more functional groups anywhere on or within the substituent. Unless otherwise specified, an alkyl group may contain any number of carbon atoms, such as for example, C₁-C₂₄, C₁-C₁₈, C₁-C₁₀, C₁-C₈, or C₁-C₆. Examples of alkyl substituents include, but are not limited to, methyl, ethyl, n-propyl, isopropyl, n-butyl, isobutyl, sec-butyl, tert-butyl, n-pentyl, iso-pentyl neo-pentyl, n-hexyl, iso-hexyl, octadecyl, dodecyl, and so forth. An alkyl substituent herein may be substituted, i.e., having one or more substituent groups appended on the alkyl group or incorporated within the alkyl chain. A substitution within the alkyl substituent chain may comprise an ether, sulfide, amino, or imine linkage, i.e., —O—, —S—, —N(R′)—, or —N═, for example, or some other intervening heteroatom(s). Examples of substitution on an alkyl substituent include, but are not limited to, —CN, —N₃, —NH₂, —NHR′, —N(R′)₂, —NO₂, —NH—NH₂, —NH—NHR′, —NH—NR′₂, -halo, —SH, —SR′, —S(═O)R′, —SO₂R′, —OPO₃²⁻, —PO₃²⁻, —OH, —OR′, —C(═O)R′, —OC(═O)R′, —CO₂R′, —NHC(═O)R′, —NR′C(═O)R′, —C(═O)NHR′, —C(═O)NR′₂, alkyl, alkenyl, cycloalkyl, heterocyclyl, and aryl, wherein each R′ above is independently selected from hydrogen —H and an alkyl moiety, including, for example, C₁₋₆ alkyl (e.g., —CH₃, —C₂H₅, -isopropyl, -tert-butyl, etc.), C₁₋₆ alkoxy (e.g., —OCH₃, —OC₂H₅), halogenated C₁₋₆ alkyl (e.g., —CF₃, —CHF₂, —CH₂F), and halogenated C₁₋₆ alkoxy (e.g., —OCF₃, —OC₂F₅). In various examples, two R′ substituents in any of these functional groups may form a ring structure.

As used herein, the term “cycloalkyl” includes any 3-, 4-, 5-, 6-, 7-, or 8-membered, saturated or unsaturated, non-aromatic carbocyclic ring, optionally substituted with one or more functional groups at any location on the cyclic substituent. Examples of cycloalkyl groups include, but are not limited to, cyclopropyl, cyclobutyl, cyclopentyl, 1-, 2-, or 5-cyclopentadienyl, cyclohexyl, 1-, 3- or 4-cyclohexenyl, 1-, 2-, or 5-(1,3-cyclohexadienyl), 1- or 3-(1,4-cyclohexadienyl), cycloheptyl, 1-, 3-, 4-, or 5-cycloheptenyl, cyclooctanyl, and so forth. Examples of substitution on an cycloalkyl substituent include, but are not limited to, —CN, —N₃, —NH₂, —NHR′, —N(R′)₂, —NO₂, —NH—NH₂, —NH—NHR′, —NH—NR′₂, -halo, —SH, —SR′, —S(═O)R′, —SO₂R′, —OPO₃²⁻, —PO₃²⁻, —OH, —OR′, —C(═O)R′, —OC(═O)R′, —CO₂R′, —NHC(═O)R′, —NR′C(═O)R′, —C(═O)NHR′, —C(═O)NR′₂, alkyl, alkenyl, cycloalkyl, heterocyclyl, and aryl, wherein each R′ above is independently selected from —H and an alkyl moiety, including, for example, C₁₋₆ alkyl (e.g., —CH₃, —C₂H₅, -isopropyl, -tert-butyl, etc.), C₁₋₆ alkoxy (e.g., —OCH₃, —OC₂H₅), halogenated C₁₋₆ alkyl (e.g., —CF₃, —CHF₂, —CH₂F), and halogenated C₁₋₆ alkoxy (e.g., —OCF₃, —OC₂F₅).

As used herein, the term “alkenyl” refers to linear or branched monovalent or divalent unsaturated hydrocarbon substituents, optionally substituted with one or more functional groups anywhere on or within the substituent. An alkenyl substituent can be viewed as being divalent if the sp² carbon is part of a molecule bearing the alkenyl substituent. An illustrative example is methylenecyclohexane, which can be viewed as cyclohexane substituted with a methylene group (i.e., a divalent alkenyl substituent, ═CH₂). Unless otherwise specified, an alkenyl group may contain any number of carbon atoms, such as for example, C₁-C₂₄, C₁-C₁₈, C₁-C₁₀, C₁-C₈, or C₁-C₆, and any degrees of unsaturation. Examples of alkenyl substituents include, but are not limited to, methylene/methylidine (=CH₂), ethylene/ethenyl (—CH═CH₂ or =CH—CH₃), propylene/propenyl (—CH₂—CH═CH₂, cis or trans —CH═CH—CH₃, =C(CH₃)₂, or cis or trans ═CH—CH₂CH₃), and so forth. An alkenyl substituent herein may be substituted, i.e., having one or more substituent groups appended on the alkenyl group or incorporated within the alkenyl chain. A substitution within the alkenyl substituent may comprise an ether, sulfide, amino, or imine linkage, i.e., —O—, —S—, —N(R′)—, or —N═, for example, or some other intervening heteroatom(s). Examples of substitution on an alkenyl substituent include, but are not limited to, —CN, —N₃, —NH₂, —NHR′, —N(R′)₂, —NO₂, —NH—NH₂, —NH—NHR′, —NH—NR′₂, -halo, —SH, —SR′, —S(═O)R′, —SO₂R′, —OPO₃²⁻, —PO₃²⁻, —OH, —OR′, —C(═O)R′, —OC(═O)R′, —CO₂R′, —NHC(═O)R′, —NR′C(═O)R′, —C(═O)NHR′, —C(═O)NR′₂, alkyl, alkenyl, cycloalkyl, heterocyclyl, and aryl, wherein each R′ above is independently selected from an alkyl moiety, including, for example, C₁₋₆ alkyl (e.g., —CH₃, —C₂H₅, -isopropyl, -tert-butyl, etc.), C₁₋₆ alkoxy (e.g., —OCH₃, —OC₂H₅), halogenated C₁₋₆ alkyl (e.g., —CF₃, —CHF₂, —CH₂F), and halogenated C₁₋₆ alkoxy (e.g., —OCF₃, —OC₂F₅). In various examples, two R′ substituents in any of these functional groups may form a ring structure.

As used herein, the terms “e-cigarette” or “ENDS” (electronic nicotine delivery system) refer generally to any electronic device capable of vaporizing a liquid composition into a vapor that can be inhaled by an individual using the device. So as not to be limiting, an “e-cigarette” herein means any design, such as a cigar, a vape pen, or any other electronic device, refillable or disposable, having a reservoir containing a vaporizable liquid therein and having the capability of vaporizing the vaporizable liquid. For simplicity, the term “electronic device” may be used to refer to any and all refillable and disposable instruments for vaping.

As used herein, the term “throat hit” refers to a sensory effect experienced at the back of the throat, perceived by an individual inhaling a vapor, such as vapor obtained from a burning cigarette or vapor delivered from an electronic device like an e-cigarette, or from dissolving a product comprising an irritant in the mouth, such as dissolving strips or lozenges, when dissolved irritant materials enter the throat. Throat hit is a type of irritating “bite” experienced at the back of the throat, which prior to the development of e-cigarettes was something not only experienced by smokers but also anticipated and enjoyed by smokers. Although subjective rather than objective, throat hit can be quantified through the use of sensory panels where participants in the panel rank the perceived throat hit on a scale of 1-10, for example, or compare throat hit between products and/or for a THR product relative to traditional cigarettes. In some examples, throat hit may be quantified as being less than, equal to, or more than the throat hit experienced from a particular cigarette in comparison. An exemplary cigarette used to standardize throat hit, such as in a sensory panel where participants rank throat hit, is Marlboro® Filter Cigarettes, Gold Pack 100's, available from Phillips IGA. As explained herein, throat hit can generally be achieved in an individual by inclusion of a chemosensory irritant in the various THR products of the present disclosure, and particularly by use of a spice additive as the chemosensory irritant. In some examples of THR products, including all pain relief THR products, a chemosensory irritant might not be included in the product.

As used herein, the term “vaping” refers to the act of using an e-cigarette or other ENDS device to inhale a vapor generated electronically from the vaporization of a liquid composition. Vaping is seen as a similar endeavor to smoking tobacco products, recognizing that an e-cigarette and vaping is not limited to nicotine. Vaping may include personal inhalation of cannabinoids, nicotine analogs, other physiologically active substances, flavorings, etc.

As used herein, the term “head rush” refers to the lightheadedness experienced by tobacco smokers and vapers that inhale nicotine, nicotine analogs, THC and other products that contain a physiologically active substance. Smokers have described this sensory experience as a ringing sensation in their heads, or a rush of adrenaline. The sensory effect is believed to be caused by nicotine or other drug in the blood and brain triggering release of adrenaline that in turn causes a tightening of the blood vessels and a temporary rise in heart rate and blood pressure of the person inhaling the active substance. This sensory experience is personal, and it varies between drug actives and between individuals because of variances in physiological tolerance. A head rush might appear within 10 seconds of inhaling nicotine, and it might last as long as 10-30 minutes. However, as a smoker or vaper continues smoking or vaping, they become more tolerant and the head rush regularly experienced becomes shorter-lasting over time. Although subjective rather than objective, head rush can be quantified through the use of sensory panels where participants in the panel rank the perceived head rush on a scale of 1-10, for example, or compare the perceived head rush between alternative products relative to traditional cigarettes. In some examples, head rush may be quantified as being less than, equal to, or more than the head rush experienced from a particular cigarette in comparison. An exemplary cigarette used to standardize throat hit, such as in a sensory panel where participants rank throat hit, is Marlboro® Filter Cigarettes, Gold Pack 100's, available from Phillips IGA. In various examples herein, THR products comprise non-nicotine alkaloids capable of stimulating nicotinic acetylcholine receptors (nAChRs), thus providing a head rush similar to inhaling nicotine.

As used herein, the term “chemosensory irritant” takes on its ordinary meaning in physiology and neuroscience, namely a substance capable of exhibiting a sensory irritation in an individual exposed to the substance. The irritation caused by a chemosensory irritant upon inhalation may be sensed, depending on the individual and the irritant, in the nose, mouth, throat, esophagus and/or lungs. The sensory effect in the throat is referred to in the definitions herein as “throat hit.” Chemosensory irritation is most studied in the context of environmental and occupation health and safety, involving such things as air pollutants and indoor environmental irritants such as formaldehyde. See, for example, “Inhalation Toxicology,” 2^{nd} Edition, H. Salem et al., editors, Taylor & Francis Group (CRC), 2006. Besides subjective measurement of chemosensory irritation, such as panelists answering symptom questionnaires, various experimental techniques can be used to study chemical-induced irritation, such as examinations of functional changes, e.g. alterations in breathing frequency and pattern, nasal, bronchial and pulmonary function parameters, nasal mucosal swelling, acoustic rhinometry, eye-blinking frequency, tear film stability and chemosensory evoked potentials, in both humans and laboratory animals (see, S. K. Kjaergaar, et al., “The Assessment of Irritation Using Clinical Methods and Questionnaires,” American Industrial Hygiene Association, 62(6):711-6, November 2001).

A chemosensory irritant optionally included in certain THR products of the present disclosure include those compounds capable of causing a physiological irritation of any one of the nose, mouth, throat, esophagus and lungs of an individual, wherein the physiological irritation is quantifiable. Examples of quantitative methods include, but are not limited to, measuring swelling of the nasal mucosa, peak airflows through the nose, acoustic rhinometry, and rhino-stereometry for measuring thickness of the anterior nasal turbinate. Questionnaires are also useful for defining a set of symptoms to characterize dose-response relationships from controlled exposure studies.

In various embodiments, a chemosensory irritant for optional use in various THR products herein is a substance capable of activating the chemosensory ion channels known as Transient Receptor Potential Channels, or TRP channels, as explained, for example, by H. J. Son, et al., “Activation of the Chemosensory Ion Channels TRPA1 and TRPV1 by Hydroalcohol Extract of Kalopanax pictus Leaves,” Biomol. Ther. (Seoul), 2012 November; 20(6): 550-555. Besides the subset of chemosensory irritants defined herein below as “spice additives,” many seemingly unrelated compounds are receptor agonists and are capable of activating one or more TRP channels (such as, A1, V1, V2, V3, V4, V5 or V6, M1, M2, M3, M4, M5, M6, M7, or M8 ion channels, and so forth), such as, for example, oleocanthal, 4-hydroxy-2-nonenal, 4-oxo-2-nonenal, allicin, allyl isothiocyanate, gingerol (included in the spice subset of irritants), icilin, polygodial, cinnamaldehyde, trans-p-methoxycinnamaldehyde, various cannabinoids, methyl syringate, 2-chlorobenzylidene malononitrile, 1-chloroacetophenone, ethyl bromoacetate, 4-hydroxyhexenal, toluene diisocyanate, p-benzoquinone, methyl p-hydroxybenzoate, flufenamic acid, niflumic acid, mefenamic acid, diclofenac, hydroxy-α-sanshool, 6-shogaol (included in the spice subset of irritants), 6-paradol, linalool, carvacrol, eugenol, thymol, vanillin, methyl eugenol, 2,6-dimethylphenol, 2,5-dimethylphenol, 3,4-dimethylphenol, 2,6-diisopropylphenol, caffeine, farnesyl thiosalicylic acid, 4-allylanisole, curcumin, capsicin (included in the spice subset of irritants), niacin, niacinamide, camphor, olvanil, and arvanil. Although nicotine is a known activator of TRPA1, it is excluded from consideration herein as a chemosensory irritant for any of the THR products in the scope of the present disclosure. See, for example, K. Talavera, et al., “Nicotine activates the chemosensory cation channel TRPA1,” Nature Neuroscience, 12, 1293-1299 (2009). One aspect of the present disclosure is to provide THR products including pain relief THR products that are expressly devoid of nicotine so that they can be used as an alternative to smoking.

Optional chemosensory irritants for use in the present THR products also include cannabinoids. In fact, certain TRP receptors are referred to as cannabinoid receptors, such CB1 and CB2. Six ion channels, TRPV1, TRPV2, TRPV3, TRPV4, TRPA1 and TRPM8 are known as ionotropic cannabinoid receptors, and thus those cannabinoids capable of binding to these receptors and activating ion channels are deemed chemosensory irritants for use in the various THR products described herein. For a review of cannabinoids and TRP ion channel activation, see C. Muller, et al., “Cannabinoid Ligands Targeting TRP Channels,” Front. Mol. Neurosci., 11, 487 (2019).

Cannabinoids (endo-, phyto- and synthetic) capable of activating a TRP ion channel, and thus usable in certain THR products of the present disclosure as chemosensory irritants include, but are not limited to, anandamide (AEA), 2-AG, NADA, OLDA, PEA, NGABA, NGly, NAsp, NSer, Δ⁹-tetrahydrocannabinol (Δ⁹-THC), Δ⁹-THCA, Δ⁹-THCV, Δ⁹-THCVA, CBD, CBDA, CBDV, CBG, CBGA, CBGV, CBN, CBC, and the synthetic cannabinoids WIN55, 212-2, AM630, (R)-AM1241, (S)-AM1241, SR141716A, Gp-1a, AM251, SR144528, JWH133, HU308, HU910, CP55,940, and Nabilone. Of particular importance herein is CBD (cannabidiol), which has been found to be the most potent and efficacious phytocannabinoid agonist of TRPV1. Also, Δ⁹-THC has been identified as the most potent phytocannabinoid for activating TRPV2, although Δ⁹-THC is not selective seeing that it also activates TRPA1. Cannabinoids may also be incorporated in THR pain relief products herein for their effect at relieving local and systemic aspects of physical pain.

Further, chemosensory irritants that activate a TRP ion channel, and thus find optional use in certain THR products herein, include numerous compounds naturally occurring in various plants, trees, shrubs, roots, flowers, fruits, seeds and nuts. Such compounds are disclosed, for example, by J. Vriens, et al., “Herbal Compounds and Toxins Modulating TRP Channels,” Curr. Neuropharmacol., 2008 March; 6(1): 79-96. In various embodiments, in addition to the “spice additives” defined and discussed herein that include such compounds as capsaicinoids, gingerols and shogaols, other TRP channel activators are of use herein and include, but are not limited to, resiniferanoids from Euphorbia reinfera, eugenol from Euenia carophyllata and Ocimum gratissiumum, ginsenosides from Panax, zingerone and paradol derived from heating or dehydrating gingerol or from Aframomum melgueta seeds, evodia compounds from Evodia rutaecarpa, various unsaturated 1,4-dialdehyde sesquiterpenes isolated from Drymis winteri, such as polygodial, isovelleral and drimanial, other 1,4-dialdehyde terpenes from Cinnamosma fragrans, such as cinnamodial, cinnamosmolide, and cinnamolide, or warbuganal from Warburgia plants. In vitro assays used to measure activation of TRP ion channels are found, for example, in R. Lehmann, et al., “Alternative in vitro assays to assess the potency of sensory irritant—Is one TRP channel enough?”, Neurotoxicology, 2017 May; 60:178-186, and J. M. Martinez, et al., Activation of TRPA1 by volatile organic chemicals leading to sensory irritation,” ALTEX 2019; 36(4): 572-582. Of further importance for use herein are the phytochemicals present in Scutellaria baicalensis (most particularly the phytochemicals baicalein, baicalin, wogonin, norwogonin, oroxylin A and β-sitosterol), Vitex agnus, Pterodon pubescens, Croton macrostachyus, Angelicae pubescentis, Ephedra sinica, Amphilophium crucigerum, Bosewellia carterii, Commiphora myrrha, Echinophora platyloba, Nypa fruticans, Zingiber officiale (presented under the spice additives subset of irritants), Corydalis saxicola, Coptis chinensis, Ononis spinosa, Parkia platycephala, Piper nigrum (presented under the spice additives subset of irritants), and Cymbopogon citratus. Chemosensory irritants for use herein can be first assessed by in vitro non-animal sensory irritation assays, followed by consumer sensory panels to evaluate subtle physiological effects and to determine dose levels and user acceptance.

As used herein, the term “spice additive” refers to an organic substance capable of producing a throat hit or other similar “hot,” “irritative,” or otherwise “burning” sensation at least at the back of the throat, and optionally in the mouth and on the lips, when the spice additive is in certain THR products used by an individual, such as a person experiencing a dissolving strip, or sucking on a lozenge or a flavored toothpick. Spice additives herein form a subset of chemosensory irritants and are known to activate one or more of the TRP ion channels. Since a group of these compounds might at first seem structurally unrelated, a “spice additive” herein can be clearly defined as an organic substance having a spiciness or “heat” in the range of from about 1,000 to about 20,000,000 Scoville Heat Units (SHU). At the lower end of the Scoville scale, compounds for use herein include, but are not limited to, gingerol (60,000 SHU), piperine (100,000 SHU) and Shogaol (160,000 SHU). In more preferred embodiments, a spice additive for optional use in certain THR products herein will have a Scoville rating of from about 100,000 to about 20,000,000 SHU, and more preferably from about 10,000,000 to about 20,000,000 SHU and will include all of the known capsaicinoids such as capsaicin (16,000,000 SHU) and dihydrocapsaicin (15,000,000 SHU), along with uncharacterized capsaicinoids that are likely isomers of the known ones. Although chemically unrelated to capsaicinoids, compounds such as piperine (100,000 SHU) and Shogaol (160,000 SHU) find use in the present compositions. A capsaicinoid spice additive for use herein includes, but is not limited to, capsaicin, dihydrocapsaicin, norcapsaicin, nordihydrocapsaicin, homocapsaicin, homodihydrocapsaicin, and mixtures thereof. An isolated capsaicinoid chemically pure or otherwise is not necessary for THR products herein, as it is also convenient to prepare THR products with an extract of a spice additive. For example, capsaicinoids can be extracted from chili peppers or other peppers using room temperature or boiling ethanol (such as in a Soxhlet extractor). The resulting red ethanol solution can then be used as is (referred to as a “capsaicin liquid” or “liquid capsaicin”) or evaporated to leave behind a thick, dark red viscous mixture of capsaicinoids (the red color believed to be from β-carotene). See, for example, F. Martins, et al., “Novel Approaches to Extraction Methods in Recovery of Capsaicin from Habanero Pepper (CNPH 15.192),” Pharmacogn. Mag. 2017 July; 13(Suppl 2): S375-S379, and Y. Zhu, “Multi-Dimensional Pungency and Sensory Profiles of Powder and Oil of Seven Chili Peppers Based On Descriptive Analysis and Scoville Heat Units,” Food Chemistry, 411, 15 Jun. 2023, Article 135488.

As used herein, the term “liquid capsaicin” or “capsaicin liquid” refers to an ethanolic or other solvent extract of a fruit obtained from a plant of the genus Capsicum annuum, such as various chili peppers. A liquid capsaicin optionally for use herein comprises about 0.9 wt. % capsaicinoids in ethanol and is available from Olive Nation, LLC, Avon, MA under the name Capsicum Flavor Extract. Such liquid extracts are easily added to alkaloid compositions used in various THR products herein.

It should be noted that “flavoring agents” are defined separately from and are entirely distinguished from “chemosensory irritants and the subset of irritants referred to herein as “spice additives.” In various examples of THR products, a flavoring agent also may be more important to incorporate into a THR product than a chemosensory irritant.

As used herein, a “flavoring agent” takes on its ordinary meaning in food science, with the caveat that a flavoring agent herein purposely excludes compounds containing a capsaicinoid, meaning that flavoring agents herein by definition have no measurable or calculable Scoville heat rating and thus are distinct from spice additive defined herein above. Flavoring agents for use herein include, but are not limited to, ethyl maltol, ethyl butyrate, ethyl acetate, maltol, ethyl vanillin, furaneol, methyl cyclopenenolone, δ-decalactone, γ-decalactone, cis-3-hexanol, iso-amyl acetate, ethyl-2-methyl butyrate, butyric acid, linalool, benzyl alcohol, ethyl hexanoate, benzaldehyde, iso-amyl isovalerate, hexanoic acid, ethyl propionate, γ-undecalactone, and hexyl acetate. Flavoring agents are more important for some THR products than others. For example, THR toothpicks may rely heavily on having a flavoring agent whereas a transdermal THR patch does not need a flavoring agent at all.

As used herein, the term “about” refers to a quantity, level, value, number, frequency, percentage, dimension, size, amount, weight or length that varies by as much as 10, 9, 8, 7, 6, 5, 4, 3, 2 or 1% to a reference quantity, level, value, number, frequency, percentage, dimension, size, amount, weight or length. For example, a quantity expressed as being “about 5 wt. %” includes a variance of up to 4.5 to 5.5 wt. %.

As used herein, the term “smoking cessation program” refers to a plan provided by a medical clinic, a dispensary, or any other business enterprise to a current smoker to help that individual stop smoking. In various embodiments, a smoking cessation program herein includes providing the individual in need thereof a THR product or combination of THR products designed to help the individual comply to the prescribed program and reduce and/or ultimately eliminate smoking and its associated tobacco harm. In certain examples, the clinic, dispensary or other business enterprise will meet with the individual placed on the cessation program on a recurring basis to interview the individual as to compliance and to supply that individual with customized THR products and combinations thereof designed to gradually move the smoker entirely to non-nicotine products. In various examples, the clinic, dispensary or other business enterprise will meet with the individual to manage perceived physical pain by prescribing combinations of THR products.

As used herein, the term “transdermal patch” refers to any adhesive assemblage configured to adhere to the skin of an individual and to deliver physiologically effective amounts of a solubilized alkaloid through the skin of the individual. The assemblage may be in the physical form of a flexible strip comprising a desired amount of solubilized alkaloid and one or more optional actives or excipients. For purposes herein, a transdermal patch may be a THR product, or a pain relief THR patch.

### General Embodiments

In various embodiments of the present disclosure, both THR products and THR pain relief products are described. Products from each category may be used separately at different times, or they may be used in combination at the same time. For example, in a combination approach to THR, an oral tobacco cessation THR product may be used by an individual while the individual is also using a THR product disclosed herein for pain relief.

Both THR products and THR pain relief products in accordance with the present disclosure share a common core technology, namely that each product comprises a substituted pyridine compound. Depending on the physical form of a THR product, a THR product may optionally comprise a chemosensory irritant.

### Tobacco Harm Reduction Products and Methods

1a. Transdermal Patch

In various embodiments, a tobacco harm reduction (THR) product in the form of a transdermal patch comprises:
at least one substituted pyridine compound of Formula (I);

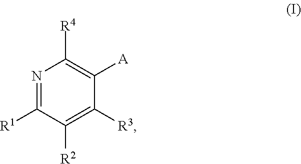

or a salt, or mixed salt thereof, wherein:
A is selected from:

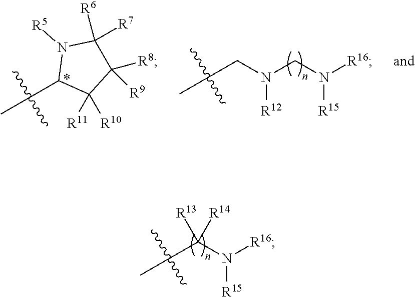

R¹, R², R³, R⁴ and R⁵ are independently selected from H, alkyl, substituted alkyl, cycloalkyl, substituted cycloalkyl, alkenyl, substituted alkenyl, and

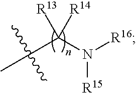

R⁶ and R⁷ are independently selected from H, alkyl, substituted alkyl, cycloalkyl, substituted cycloalkyl, alkenyl and substituted alkenyl, or R⁶ and R⁷ together are ═O;
R⁸, R⁹, R¹⁰, R¹¹, R¹², R¹³, and R¹⁴ are independently selected from H, alkyl, substituted alkyl, cycloalkyl, substituted cycloalkyl, alkenyl, and substituted alkenyl;
n is an integer from 1 to 10; and
R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered optionally substituted heterocyclic ring,
with the proviso that if A is

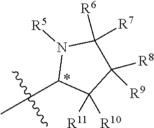

and R⁵ is methyl, then R¹, R², R³, R⁴, R⁶, R⁷, R⁸, R⁹, R¹⁰, and R¹¹ cannot all be H; and
wherein the substituted pyridine compound and the solubilizer comprise a solubilized alkaloid mixture disposed on a surface of the substrate, and
wherein the solubilized mixture is entirely devoid of (R) or (S) nicotine.

In various embodiments, the tobacco harm reduction (THR) product further comprises at least one solubilizer; and/or a substrate.

In various embodiments, the solubilized substituted pyridine compound further comprises at least one organic acid, such that the molar ratio of moles substituted pyridine compound to moles total organic acid is from about 25:1 to about 75:1. Organic acids for use herein are described below and in the definitions.

In various examples, the amount by weight of substituted pyridine compound per patch is from about 1 mg to about 50 mg. Sufficient solubilizer is used to solubilize the desired amount of alkaloid into an oily mixture or paste amenable to layering onto a surface of the substrate.

In various embodiments, the substrate measures about 1-inch×1-inch square, up to about 2-inches×2-inches square, and less than about ⅛^{th} inch in thickness. Other shapes such as round or rectangular and other practical sizes for use on skin are anticipated.

In various embodiments, the substrate comprises a swatch of fabric, nonwoven, or plastic, rubber or latex sheet material. In some examples, an adhesive is provided around outer portions of the swatch and the solubilized mixture is provided near the center of the swatch, on the same side and spaced apart from the adhesive edging.

In various embodiments, the solubilizer is selected from partially hydrogenated, or fully hydrogenated vegetable oils such as soybean oil, olive oil, sesame oil, palm oil, coconut oil, and the like. Solubilizers may also be fractionated cuts of fatty acids, fatty acid mixtures, mono-, di-, or triglycerides of fatty acids, wherein the fatty acids and/or glycerides may have a particular chain length distribution (e.g., short-, medium-, or long-chain fatty acids). Of particular use herein are “medium-chain triglycerides” (or “MCT's”), which refers to the glycerin triesters of fatty acids having from about 6 to about 12 carbon atoms, such as caprylic acid, caproic acid, lauric acid, and/or capric acid in any combination, (i.e., C₆-C₁₂ fatty acid triglycerides). In various examples, medium-chain triglycerides can be obtained by fractionating palm kernel oil and coconut oil. Medium-chain triglycerides, USP/NF, CAS No. 73398-61-5, for use in the transdermal THR compositions herein is available, for example, from Specialized Rx Products, LLC, Circle Pines, MN. Also of use herein is Organic MCT Oil-Fractionated Coconut Oil (caprylic/capric triglyceride), CAS Nos. 73398-61-5 and 65381-09-1, having a specific gravity of about 0.930-0.960 g/mL (typically on average, 0.951 g/mL).

In various embodiments, the transdermal patch further comprises a permeation enhancer in the solubilized alkaloid mixture.

In various embodiments, transdermal patches herein are prepared by methods well known in the art, limited only by the alkaloid load capacity of the respective transdermal technology employed.

For example, one common transdermal technology comprises a polymer matrix design wherein the polymer matrix can be prepared using natural polymers including, for example and without limitation, cellulose derivatives, zein, gelatin, shellac, waxes, proteins, gums and their derivatives, natural rubber, starch and the like; synthetic elastomers including, for example and without limitation, polybutadiene, polyisobutylene, hydrin rubber, polysiloxane and other silicone pressure sensitive adhesives available from Dow Corning (Elizabethtown, KY), silicone rubber, nitrile-containing compounds, acrylonitrile, butyl rubber, styrene-butadiene rubber, neoprene and the like; and/or synthetic homopolymers or copolymers including, for example and without limitation, polyvinyl alcohol, polyvinyl chloride, polyethylene, polypropylene, polyacrylate, polystyrene, polyamide, polyurea, polyvinylpyrrolidone, polymethylmethacrylate, epoxy, polyethylene/vinyl acetate, and the like.

To this matrix is applied and/or incorporated at least one substituted pyridine compound according to Formula (I) and, in some examples, penetration enhancers, at least one solvent, and/or at least one surfactants. An adhesive is used to affix the substrate portion of the transdermal patch to the skin of an individual. Such adhesives can be affixed as a layer to the substrate, or as one of the ingredients used to prepare the matrix, and the matrix applied as a layer on the patch substrate. Other matrix technologies can include crystallization inhibitors and other acceptable excipients. Matrix technology can also be developed into single or multi-layer patches. See, for example, U.S. Pat. Nos. 5,474,783; 5,958,446; and 6,440,454, and PCT Application Publication No. WO 97/43989, each of which are incorporated herein by reference.

Also well known in the transdermal patch art is a reservoir system, typically characterized by the inclusion of a reservoir, defined as a compartment containing solubilized or suspended substituted pyridine compound, which is separate and distinct from a release liner. Various adhesive systems are also available for reservoir-type systems. Reservoir systems are frequently the system of choice when substantial alkaloid loads are required. See, for example, U.S. Pat. Nos. 7,387,789; 5,656,286; 8,114,434; 6,024,976; and U.S. Patent Publication Nos.: 2012/0269878 and 2012/0283671, each of which are incorporated herein by reference.

Other transdermal patch technologies include, but are not limited to, iontophoresis, electroporation, ultrasound/sonophoresis, and microscopic projection (e.g., microneedles) (see, e.g., Patel, D., et al., The Pharma Innovation, Transdermal Drug Delivery System: A Review, Vol. 1, No. 4, pg. 66-75 (2012)). Any of these and other transdermal systems can be used for the preparation of solubilized alkaloid transdermal patches.

1b. Dissolving Films

In various embodiments, a THR dissolving film is a mucoadhesive film, further comprising a substituted pyridine compound in combination with water-soluble and/or water-dispersible polymers, polyols, surfactants, and other ingredients. Optionally, a THR dissolving film comprises a chemosensory irritant such as a spice additive, so as to provide a throat hit or other oral experience. Optionally, a THR dissolving film comprises a flavoring agent.

In various embodiments, a mucoadhesive film configured for oral use comprises:
at least one substituted pyridine compound of Formula (I);

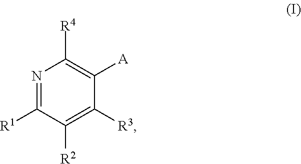

or a salt, or mixed salt thereof, wherein:
A is selected from:

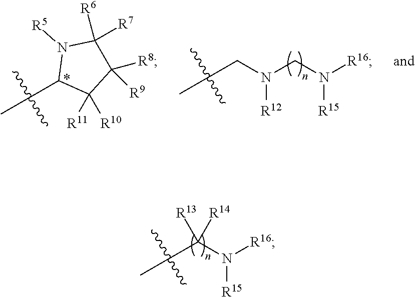

R¹, R², R³, R⁴ and R⁵ are independently selected from H, alkyl, substituted alkyl, cycloalkyl, substituted cycloalkyl, alkenyl, substituted alkenyl, and

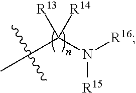

R⁶ and R⁷ are independently selected from H, alkyl, substituted alkyl, cycloalkyl, substituted cycloalkyl, alkenyl and substituted alkenyl, or R⁶ and R⁷ together are ═O;
R⁸, R⁹, R¹⁰, R¹¹, R¹², R¹³, and R¹⁴ are independently selected from H, alkyl, substituted alkyl, cycloalkyl, substituted cycloalkyl, alkenyl, and substituted alkenyl;
n is an integer from 1 to 10; and
R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered optionally substituted heterocyclic ring,
with the proviso that if A is

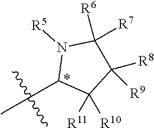

and R⁵ is methyl, then R¹, R², R³, R⁴, R⁶, R⁷, R⁸, R⁹, R¹⁰, and R¹¹ cannot all be H; and
wherein the mucoadhesive film is entirely devoid of (R) or (S) nicotine.

In various embodiments, mucoadhesive film further comprises at least one polymer, at least one polyol, optionally, at least one surfactant, optionally, at least one chemosensory irritant and combinations thereof.

In various embodiments, the dissolving film further comprises at least one organic acid, such that the molar ratio of moles substituted pyridine compound of Formula (I) to moles total organic acid is from about 25:1 to about 75:1. Organic acids for use herein are described below and in the definitions.

In preferred examples, a mucoadhesive film in the form of a small strip is configured to dissolve entirely in the oral cavity of an individual, either instantly or over the course of no more than about 15-30 seconds. Each small strip may contain from about 1 mg to about 10 mg substituted pyridine compound according to Formula (I).

In various embodiments, a polyol for use herein includes, but is not limited to, glycerin, 1,2-propylene glycol, 1,3-propylene glycol, polysaccharides, and sugar alcohols. Exemplary polysaccharides include pullulan and carrageenan.

In various embodiments, a polymer for use in a mucoadhesive film herein include various hydrophilic and water-dispersible gums, cellulosic materials, starches and synthetic polymers.

In various embodiments, polymers for use herein include, but are not limited to, agar, acacia, agarose, albumin, alginate, Arabic gum, casein, chitin, chondroitin, dextrin, fibroin, fucoidan, galactan, gellan, guar, scleroglucan, pullulan, xyloglucan, pectin, xanthan, tragacanth, psyllium, cellulose, cellulose acetate, hyaluronan, elastin-like polypeptides, β-cyclodextrin, collagen, gelatin, chitosan, carrageenan, polylactic acid, polyglycolic acid, poly(lactic-glycolic acid) (PLGA), poly(2-hydroxyethyl methacrylate), poly(2-hydroxypropyl methacrylate), poly(acrylic acid), carboxymethyl cellulose, carboxyethyl cellulose, hydroxyethyl cellulose, hydrophobically-modified hydroxyethyl cellulose, hydroxypropyl cellulose, hydroxypropyl methyl cellulose, methyl cellulose, ethyl cellulose, microcrystalline cellulose, nitrocellulose, polyvinyl alcohol, polyvinylpyrrolidone, polyvinyl methacrylate, carboxyvinyl polymers, polyvinyl acetate, polyvinyl co-polymers, starch, modified starches, polyethylene glycol (PEG), and mixtures thereof.

A mucoadhesive film according to the present disclosure comprises from about 20 wt. % to about 75 wt. % polymer, and more preferably between 50 wt. % and about 75 wt. % polymer, based on the total weight of the film.

In various embodiments, a mucoadhesive film herein may optionally comprise at least one surfactant, such as one or more nonionic surfactants. Surfactants include, but are not limited to, sugar esters, such as, for example, sorbitan fatty acid esters sold under the trade name Span®, and polyethoxylated (POE) sorbitan fatty acid esters sold under the trade name Tween®. Of particular interest herein is polysorbate 80, C₆₄H₁₂₄O₂₆ (available as Tween® 80) which is a sorbitan monooleate ethoxylated with a total of about 20 (POE) units. Other polysorbates, such as lauryl esters rather than oleic esters, other (POE) levels, also find use herein. Polysorbates and sorbitan esters suitable for use as surfactants in mucoadhesive films herein are available, for example, from BASF Pharma, Florham Park, NJ.

In various embodiments, a THR mucoadhesive film may further comprise a plasticizer.

A THR mucoadhesive film according to the present disclosure can be prepared as follows. The polyalcohol, optional surfactants and plasticizers, and other ingredients except the polymer are dissolved in a sufficient amount of a compatible solvent such as water, alcohol or mixtures thereof. After a clear solution has been formed, the polymer or mixture of polymers is slowly added with stirring, with heating as necessary, until a clear and homogeneous solution has been formed, followed by addition of the chosen substituted pyridine compound of Formula (I) and flavors, preservatives, etc. The resulting solution is evenly coated onto a suitable carrier material, such as with a blade, and dried to form a film. The carrier material preferably has a surface tension allowing the polymer solution to spread and dry and to be ultimately removable therefrom. Examples of suitable substrate materials include non-siliconized polyethylene terephthalate film, non-siliconized kraft paper, polyethylene-impregnated kraft paper, and non-siliconized polyethylene film. The coating of the polymer solution onto the carrier material can be performed using any conventional coating equipment, such as a knife-over-roll coating method.

The thickness of the resulting THR mucoadhesive film depends on the concentration of alkaloid in the coating solution and is preferably from about 5 to about 200 μm thick. Drying of the film is carried out at ambient temperatures or with heating. For a suitable mouth feel, a THR mucoadhesive film herein is preferably <70 μm thick. To obtain final THR dissolving films, the dry film is cut into pieces of suitable size and shape (e.g., 0.5 inch×0.75 inch each) and packed into a suitable container such as a small plastic case with a snap lid.

In various examples, a THR dissolving film strip may further comprise any combination of chemosensory irritant, flavoring agent, sweetener, stabilizer, or preservative, including antioxidant. These additional ingredients are detailed further below, being optional to many of the THR product forms.

1c. Lozenges

In various embodiments, a THR product for oral administration comprises a dissolvable lozenge. In certain examples, a THR lozenge resembles a typical cough lozenge, intended to dissolve slowly in the mouth of an individual, such as over the course of several minutes.

In various examples, a THR lozenge comprises:
at least one substituted pyridine compound of Formula (I);

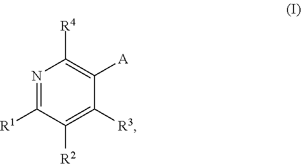

or a salt, or mixed salt thereof, wherein:
A is selected from:

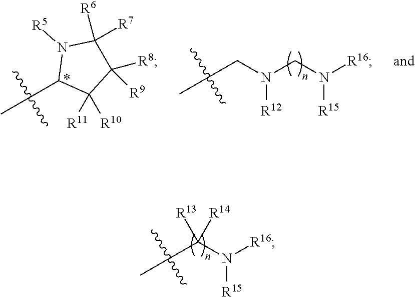

R¹, R², R³, R⁴ and R⁵ are independently selected from H, alkyl, substituted alkyl, cycloalkyl, substituted cycloalkyl, alkenyl, substituted alkenyl, and

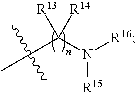

R⁶ and R⁷ are independently selected from H, alkyl, substituted alkyl, cycloalkyl, substituted cycloalkyl, alkenyl and substituted alkenyl, or R⁶ and R⁷ together are ═O;
R⁸, R⁹, R¹⁰, R¹¹, R¹², R¹³, and R¹⁴ are independently selected from H, alkyl, substituted alkyl, cycloalkyl, substituted cycloalkyl, alkenyl, and substituted alkenyl;
n is an integer from 1 to 10; and
R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered optionally substituted heterocyclic ring,
with the proviso that if A is

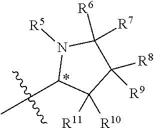

and R⁵ is methyl, then R¹, R², R³, R⁴, R⁶, R⁷, R⁸, R⁹, R¹⁰, and R¹¹ cannot all be H; and
wherein the lozenge is entirely devoid of (R) or (S) nicotine.

In various examples, the THR lozenge further comprises a binder, a sweetener, water and combinations thereof.

In various examples, a THR lozenge comprises from about 0.5 wt. % to about 20 wt. % binder; from about 75 wt. % to about 90 wt. % by weight sweetener; and from about 10 wt. % to about 20 wt. % water, based on the total weight of the lozenge.

In various embodiments, the binder is selected from natural gums such as gum Arabic or gum tragacanth, gelatin, pectin, starch, and mixtures thereof. The amount of binder is from about 0.5 wt. % to about 20 wt. %, preferably from about 0.5 wt. % to about 8 wt. %, based on the total weight of the lozenge.

In various embodiments, the sweetener is selected from dextrose, glucose, sucrose, and mixtures thereof. Dextrose can be a substitute for the traditionally used sucrose.

In various embodiments, lozenges further comprise at least one organic acid, such that the molar ratio of moles substituted pyridine compound of Formula (I) to moles total organic acid is from about 25:1 to about 75:1. Organic acids for use herein are described below and in the definitions.

THR lozenges according to the present disclosure are made by a process comprising (1) mixing of the substituted pyridine compound of Formula (I) and sweetener with at least one binder in solution, (2) kneading, rolling, stamping of the resulting paste to produce lozenges, and (3) drying of the lozenges obtained. In certain examples, the binder is introduced in several portions into the sweetener and alkaloid mixture.

In certain examples, the sweetener/alkaloid mixture is introduced into an optionally heated kneader having zig-zag paddles. The binder in solution is then added to the sweetening component. Adding the binder in several portions provides a more acceptable surface condition. The binder can be added in two portions. The paste can further include flavors, additional substituted pyridine compounds, colorants and optionally organic acids as described above to partially form alkaloid salts. The composition is intimately mixed and the paste is then rolled, stamped and dried in an oven at 30-50° C. for 8 to 24 hours to produce alkaloid lozenges.

In various examples, a THR lozenge may further comprise any combination of chemosensory irritant, stabilizer, or preservative, including antioxidant. These additional ingredients are detailed further below, being optional to many of the other THR product forms.

1d. Chewing Gum

In various embodiments, a THR product is provided in the form of a chewing gum. For purposes herein, a chewing gum can be any type of product, such as a candy coated gum, small beads, flat sticks of gum, bubble gum, shredded bubble gum that resembles chewing tobacco, paper wrapped short sticks of gum resembling cigarettes, and so forth.

In various embodiments, a THR chewing gum comprises:
at least one substituted pyridine compound of Formula (I);

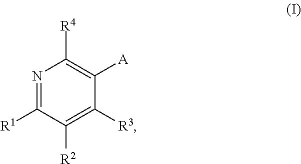

or a salt, or mixed salt thereof, wherein:
A is selected from:

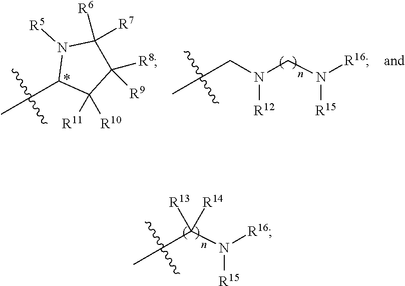

R¹, R², R³, R⁴ and R⁵ are independently selected from H, alkyl, substituted alkyl, cycloalkyl, substituted cycloalkyl, alkenyl, substituted alkenyl, and

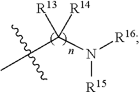

R⁶ and R⁷ are independently selected from H, alkyl, substituted alkyl, cycloalkyl, substituted cycloalkyl, alkenyl and substituted alkenyl, or R⁶ and R⁷ together are ═O;
R⁸, R⁹, R¹⁰, R¹¹, R¹², R¹³, and R¹⁴ are independently selected from H, alkyl, substituted alkyl, cycloalkyl, substituted cycloalkyl, alkenyl, and substituted alkenyl;
n is an integer from 1 to 10; and
R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered optionally substituted heterocyclic ring,
with the proviso that if A is

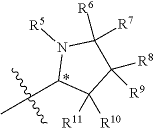

and R⁵ is methyl, then R¹, R², R³, R⁴, R⁶, R⁷, R⁸, R⁹, R¹⁰, and R¹¹ cannot all be H; and
a water-insoluble chewing gum base,
wherein the chewing gum is entirely devoid of (R) or (S) nicotine.

In various embodiments, THR chewing gum in accordance with the present disclosure further comprises at least one organic acid, such that the molar ratio of moles substituted pyridine compound of Formula (I) to moles total organic acid is from about 25:1 to about 75:1. Organic acids for use herein are described below and in the definitions.

In certain examples, the water-insoluble gum base may comprise elastomer, plasticizer, filler, texturizer such as a softening agent, oil, thickener, surfactant, stabilizer, colorant, and flavoring agent. Water-insoluble gum bases for use herein are disclosed, for example, in U.S. Pat. Nos. 3,052,552 and 2,197,719.

Elastomers include, for example, polyisobutylene, isobutylene isoprene copolymers, styrene butadiene copolymers, polyvinyl acetate, polyisoprene, polyethylene, vinyl acetate vinyl laurate copolymers, natural rubber, and combinations thereof.

The water-insoluble gum base can comprise elastomeric plasticizers. These include, for example, natural rosin esters.

Additionally, the water-insoluble gum base can include fillers, texturizers, softeners and emulsifiers. Softeners optimize how chewable a gum is and optimize mouth feel of the gum. Softeners/emulsifiers that are typically used include, for example, tallow, hydrogenated tallow, hydrogenated and partially hydrogenated vegetable oils, cocoa butter, glycerol monostearate, glycerol triacetate, lecithin, and combinations thereof.

In addition to a water insoluble gum base portion, a THR chewing gum composition herein includes a water soluble portion. The water soluble portion can include a substituted pyridine compound of Formula (I), emulsified in an oil for example, sweeteners, flavoring agents, stabilizers, softeners, surface active agents, emulsifiers, colors, acidulants, fillers, antioxidants, and other components that optimize user experience.

In various embodiments, the water-insoluble gum base may be obtained commercially, such as from Dreyfus, South Plainfield, N. J, under the tradename Dreyco®.

In a nonlimiting example, a THR chewing gum comprises from about 0.05 wt. % to about 1 wt. % substituted pyridine compound according to Formula (I); 15 wt. % to 70 wt. % Dreyco® gum base; 27 wt. % to 82 wt. % sorbitol and/or mannitol; 0.5 wt. % to 1 wt. % flavoring agent; and 0.2 wt. % to 1 wt. % of an oil.

1e. Oral Pouches

In various embodiments, substituted pyridine compounds of Formula (I) in accordance with the present disclosure also find use in oral alkaloid dispensing pouches. A pouch for oral use comprises a relatively small (e.g., 0.5 in×0.75 in) saliva-permeable fabric material enclosure containing a powder sealed therein, capable of releasing an alkaloid into the oral cavity of an individual when the pouch is placed sublingually or in the buccal cavity of the individual. An alkaloid dispensing pouch according to the present disclosure is configured to release a substituted pyridine compound of Formula (I) over time. In some embodiments, release of an alkaloid into the oral cavity of an individual from an alkaloid dispensing pouch may be characterized as “time release.”

In various embodiments, an alkaloid dispensing pouch comprises a saliva-permeable nonwoven fabric and a powdered alkaloid composition sealed therein. In certain examples, the powdered alkaloid composition sealed within the pouch comprises an admixture of crystalline or other solid form of substituted pyridine compound according to Formula (I) and a solid carrier, or a substituted pyridine compound according to Formula (I) as an oil, pure or otherwise, adsorbed onto a solid carrier. In either scenario, the mixture is preferably a powder or granulate. In other embodiments, a powdered alkaloid composition within an alkaloid dispensing pouch comprises a substituted pyridine compound according to Formula (I), a solvent, and a carrier, wherein the alkaloid dissolved in the solvent is adsorbed onto the carrier.

### 1e. (1) General Embodiments of Alkaloid Dispensing Pouches:

In general embodiments, an alkaloid dispensing pouch adapted for release of an alkaloid therefrom into the oral cavity of an individual, comprises:

a saliva-permeable nonwoven fabric defining an enclosure containing an alkaloid composition therein, said alkaloid composition comprising:

at least one substituted pyridine compound, or salt, or mixed salt thereof, according to Formula (I):

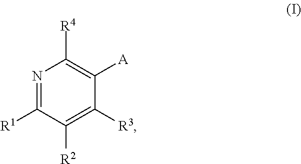

wherein:
A is selected from:

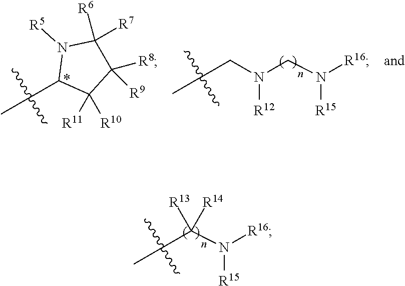

R¹, R², R³, R⁴ and R⁵ are independently selected from H, alkyl, substituted alkyl, cycloalkyl, substituted cycloalkyl, alkenyl, substituted alkenyl, and

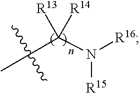

R⁶ and R⁷ are independently selected from H, alkyl, substituted alkyl, cycloalkyl, substituted cycloalkyl, alkenyl and substituted alkenyl, or R⁶ and R⁷ together are ═O;
R⁸, R⁹, R¹⁰, R¹¹, R¹², R¹³, and R¹⁴ are independently selected from H, alkyl, substituted alkyl, cycloalkyl, substituted cycloalkyl, alkenyl, and substituted alkenyl;
n is an integer from 1 to 10; and
R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered optionally substituted heterocyclic ring,
with the proviso that if A is

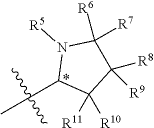

and R⁵ is methyl, then R¹, R², R³, R⁴, R⁶, R⁷, R⁸, R⁹, R¹⁰, and R¹¹ cannot all be H; and
wherein the alkaloid dispensing pouch is entirely devoid of (R) or (S) nicotine.

In various embodiments, the an alkaloid dispensing pouch further comprises a carrier.

In various embodiments, the alkaloid composition is an admixture of a substituted pyridine compound of Formula (I), in either a solid or oil form, and the carrier.

In various embodiments, the carrier comprises microcrystalline cellulose.

In various embodiments, the alkaloid composition optionally comprises a chemosensory irritant and/or a flavoring agent.

In various embodiments, the alkaloid composition disposed in a fabric pouch further comprises at least one organic acid, such that the molar ratio of moles substituted pyridine compound of Formula (I) to moles total organic acid is from about 25:1 to about 75:1. Organic acids for use herein are described below and in the definitions.

In various embodiments, the alkaloid composition further comprises a solvent, such that the alkaloid composition can be adsorbed as a solution onto the carrier to form a powdered or granular composition that can be filled into said enclosure. In certain examples, the solvent is selected from the group consisting of propylene glycol, glycerin, water, ethanol, and mixtures thereof.

### 1e. (2) Saliva-Permeable Fabric for Pouches:

Pouch material for use herein is chosen for its insolubility in water and for its permeability to water and saliva. Material is sized such that a filled pouch is appropriate for sublingual or buccal placement for extended periods of time. Subjectively, an alkaloid dispensing pouch is configured for comfortable mouth feel, including size of the filled and sealed pouch and texture of the pouch fabric.

Pouch material for use herein may be constructed from any number of various water-insoluble nonwoven materials, called “fabrics”. Nonwoven fabrics, with their multitude of uses, are well known to those skilled in textiles. Nonwovens are described very thoroughly in “Nonwoven Fabrics: Raw Materials, Manufacture, Applications, Characteristics, Testing Processes,” editors W. Albrecht, H. Fuchs and W. Kittelmann, Wiley-VCH Verlag GmbH & Co. KgaA Weinheim, 2003. Such material can be prepared by forming a web of continuous filament and/or staple fibers and optionally bonding these fibers at fiber-to-fiber contact points to provide fabrics with the desired properties. The term “bonded nonwoven fabric” is used to include nonwoven fabrics where a major portion of the fiber-to-fiber bonding is achieved by either thermal fusion of adjacent fibers, or adhesive bonding that is accomplished through incorporation of adhesives in the web to “glue” fibers together, or by other bonding such as obtained by the use of liquid or gaseous bonding agents (usually in conjunction with heating) to render the fibers cohesive. Chemical bonding may be accomplished through the use of adhesive or latex powders dispersed between the fibers in the web, which is then activated by heat, ultraviolet or infrared radiation, or other suitable activation method. Thermally and chemically bonded carded webs are described in U.S. Pat. No. 6,689,242 to Bodaghi, the subject matter of which is incorporated herein. Thermally and/or chemically bonded nonwovens may be used as the pouch material herein. Powder bonding is a dry process that starts with the carding of staple fibers to form a fibrous web, which is then treated with powdered thermal plastic adhesive or latex materials and subjected to a series of ovens and calendar rolls to produce the nonwoven.

Nonwovens may also comprise fibers known as “bi-component fibers”, for example “sheath/core bi-component fibers”, which are fibers having an outer sheath area or layer with a lower melting point than the inner core area, allowing for efficient and controlled thermal bonding through melting of just the outer layer of each fiber. That is, the outer surface of a bi-component fiber can be made to have a lower melting point than the core of the fiber. For example, binder bi-component fibers where one component has adhesive properties under bonding conditions are widely employed to provide integrity to fibrous webs used as absorbents in personal care products or in filtration products. Additionally, multi-component fibers are similarly known and commercially incorporated into nonwovens. Examples of Such multi-component fibers are described in U.S. Pat. No. 5,382,400 (Pike et al.) and U.S. Pat. No. 5,866,488 (Terada et al.) and incorporated herein in their entireties.

During the bonding of the fibers, the web may be simultaneously subjected to mechanical compression to obtain the desired bonding, weights and thicknesses in a process known as “thermal compression bonding.” Thermal compression bonding may be accomplished by using a hot embossing roll and a heat flat calendar roll and incorporating a method in which a heat treating machine such as a hot blast-circulating type, a hot through-air type, an infrared heater type or a vertical hot blast-blowing type is used to carry out thermal compression bonding. Mechanical compression may be used to set the loft or thickness of fabrics with similar basis weights. Normally, increasing the basis weight, or the mass per square area increases thickness, and increasing bonding and compression, decreases loft.

Nonwoven webs may be formed from a number of processes, for example, melt-blown, spun-bonded or spun-laid, toe-opened, wet-laid, air-laid, carded, and high pressure hydro-entangled. The basis weight of nonwoven webs is usually expressed in ounces of material per square yard (osy) or grams per square meter (gsm) and the fiber diameters are usually expressed in microns, or in the case of staple fibers, “denier.” Denier is defined as grams per 9000 meters of fiber length. For a fiber having circular cross-section, denier may be calculated as fiber diameter in microns squared, multiplied by the density in grams/cc, multiplied by 0.00707. A lower denier indicates a finer fiber and a higher denier indicates a thicker or heavier fiber. The “mean fiber denier” is the sum of tile deniers for each fiber, divided by the number of fibers. A distribution of deniers or an “average fiber denier” refers to a distribution of fiber diameters around a specific value. As used herein, the term “bulk density” refers to the weight of a material per unit of volume and usually is expressed in units of mass per unit of bulk volume (e.g., grams per cubic centimeter). Nonwovens may be produced by fibers having a single average value of diameters or denier, or two or more average value diameter fibers may be used together. For example, two or more distributions of fiber deniers may be combined into separate fiber webs (2½ denier and 4 denier fibers carded together for example). Then separate fiber webs may be laminated together. For example, a single nonwoven may comprise 2½, 4, 6, and 15 denier fibers, meaning it was constructed with four separate denier fibers (four separate average diameters of fibers.

“Spun-bonded fibers” refers to fibers formed by extrusion of molten thermoplastic material as filaments, described for example in U.S. Pat. No. 4,340,563 to Appel; U.S. Pat. No. 3,692,618 to Dorschner; U.S. Pat. No. 3,802,817 to Matsuki; U.S. Pat. Nos. 3,338,992 and 3,341,394 to Kinney; U.S. Pat. No. 3,502,763 to Hartman; U.S. Pat. No. 3,542,615 to Dobo; and, U.S. Pat. No. 5,382,400 to Pike, the entire contents of each incorporated herein by reference. Spun-bond fibers are generally not tacky when they are deposited onto a collecting surface. Spun-bond fibers are generally continuous and have average diameter from about 7 microns to about 60 microns, and most often between about 15 and 25 microns.

“Melt-blown” refers to fibers formed by extruding molten thermoplastic material through a plurality of fine, normally circular, die capillaries as molten threads or filaments into converging high velocity, usually hot, gas/air streams that attenuate the filaments of molten thermoplastic material to reduce their diameter, which may end up at micro-fiber diameter. Thereafter, the melt-blown fibers are carried by tile high velocity gas stream and are deposited on a collecting surface to form a web of randomly dispersed fibers. Such a process is disclosed, for example, in U.S. Pat. No. 3,849,241 (Butin et al.). Melt-blown fibers are micro-fibers that may be continuous or discontinuous and are generally smaller than 10 microns in average diameter and are generally tacky when deposited onto a collecting surface.

“Air-laid” is a well-known process by which a fibrous nonwoven layer can be formed. In the air-laid process, bundles of small fibers having typical lengths of from about 3 to about 52 millimeters are separated and entrained in an air supply and deposited onto a forming screen, usually with the assistance of a vacuum. The randomly deposited fibers then are bonded to one another using, for example, hot air to activate a binder component or latex adhesive. The air-laying process is taught in, for example, U.S. Pat. No. 4,640,810 to Laursen and U.S. Pat. No. 5,885,516 to Christensen.

The preferred fibers incorporated in pouch fabric for use herein may be single-, bi- (e.g., sheath/core), or multi-component fibers, made from; poly-olefins such as polypropylene, polyethylene; various polyesters such as poly(ethylene terephthalate)-PET, poly(butylene terephthalate)-PBT or poly(trimethylene terephthalate)-PTT, polycarbonates, or polybutyrates and tile like; viscose rayon; various polyamides such as nylons; polyacrylates; and, modacrylics, and mixtures of these types of polymers. In various preferred examples, viscose is used as the saliva-permeable nonwoven pouch fabric for alkaloid dispensing pouches herein.

Filling and sealing methods for pouches comprising nonwoven fabrics are disclosed in U.S. Patent Application Publication US 2023/0211907, published Jul. 6, 2023 and assigned to Swedish Match North Europe AB, incorporated herein by reference. In various embodiments, an alkaloid dispensing pouch herein comprises a fill weight of an alkaloid composition (dry powder or granulate) of from about 100 mg to about 500 mg, preferably about 200 mg to about 400 mg. An upper limit is reached when pouches no longer fit comfortably sublingually or within the buccal cavity. Since the weight of the small piece of nonwoven fabric is negligible compared to the weight of an individual pouch, the fill weight of an alkaloid dispensing pouch herein is from about 100 mg to about 500 mg dry powdered or granulated alkaloid composition per pouch.

In various embodiments, an alkaloid dispensing pouch measures from about 0.12 inches to about 0.50 inches in width (or diameter) and from about 0.25 to about 0.75 inches in height. In some examples, a pouch may look like a small pillow (square, for example), or more like a capsule.

### 1e. (3) Powdered Fill Compositions for Alkaloid Dispensing Pouches:

In various embodiments, alkaloid dispensing pouches herein comprise a powdered or granular composition further comprising a substituted pyridine compound of Formula (I) admixed with a carrier or as a liquid composition comprising a substituted pyridine compound of Formula (I) dissolved in a solvent and adsorbed onto a carrier. Either route is intended to result in a dry powdered or granulated alkaloid fill material to seal inside a nonwoven fabric enclosure.

Carriers for use herein include, but are not limited to, agar, agarose, albumin, alginate, casein, chitin, chondroitin, dextrin, fibroin, fucoidans, galactans, gellan, guar, scleroglucan, pullulan, xyloglucan, pectin, xanthan, psyllium, silica gel, fumed silica, magnesium aluminum silicates, clay, bentonite, hectorite, mesoporous silica, cellulose, cellulose acetate, hyaluronan, various elastin-like polypeptides, β-cyclodextrin, collagen, gelatin, chitosan, carrageenan, polylactic acid, polyglycolic acid, poly(lactic-glycolic acid) (PLGA), poly(2-hydroxyethyl methacrylate), poly(2-hydroxypropyl methacrylate), poly(acrylic acid), carboxymethyl cellulose, carboxyethyl cellulose, hydroxyethyl cellulose, hydrophobically-modified hydroxyethyl cellulose, hydroxypropyl cellulose, hydroxypropyl methyl cellulose, methyl cellulose, ethyl cellulose, microcrystalline cellulose, nitrocellulose, polyvinyl alcohol, polyvinylpyrrolidone, polyvinylmethacrylate, carboxyvinyl polymers, polyvinylacetate, polyvinyl co-polymers, various starches, modified starches, and combinations thereof. See, S. M. Fijul Kabir, et al., “Cellulose-based hydrogel materials: chemistry, properties and their prospective applications,” Prog. Biomater., 7, 153-174 (2018).

Carriers may be chosen on the basis of absorptive capacity and, to a lesser extent, taste. In instances where a carrier does not dissolve at all in the saliva present in the oral cavity, taste will not be a factor, and it is unlikely any insoluble carrier will pass between fibers of a nonwoven. In other examples, a carrier may be soluble, and will pass through the porous pouch fabric along with the alkaloid composition. In preferred embodiments, a carrier comprises microcrystalline cellulose.

The resulting powdered or granular alkaloid fill material is filled into nonwoven pouches at fill levels of from about 100 mg to about 500 mg dry powdered or granulated alkaloid composition per pouch and subsequently sealed to make individual alkaloid dispensing pouches for oral use.

1f. Impregnated Toothpicks

In various embodiments, a THR product for oral use comprises impregnated toothpicks. In many regards, THR impregnated toothpicks resemble familiar cinnamon toothpicks, but are not necessarily cinnamon flavored as countless flavoring agents can be used. Cinnamon toothpicks generally comprise a sliver of wood impregnated with cinnamon oil.

In various examples herein, a THR toothpick comprises a sliver of wood impregnated with an alkaloid composition comprising at least one substituted pyridine compound, or salt, or mixed salt thereof, according to Formula (I):

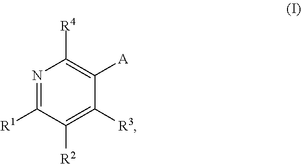

wherein:
A is selected from:

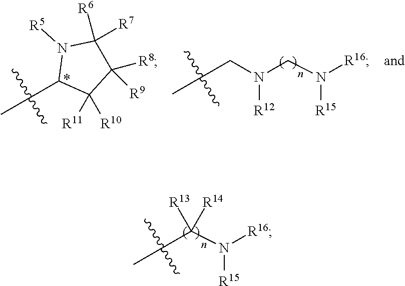

R¹, R², R³, R⁴ and R⁵ are independently selected from H, alkyl, substituted cycloalkyl, substituted cycloalkyl, alkenyl, substituted alkenyl, and

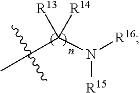

R⁶ and R⁷ are independently selected from H, alkyl, substituted alkyl, cycloalkyl, substituted cycloalkyl, alkenyl and substituted alkenyl, or R⁶ and R⁷ together are ═O;
R⁸, R⁹, R¹⁰, R¹¹, R¹², R¹³, and R¹⁴ are independently selected from H, alkyl, substituted alkyl, cycloalkyl, substituted cycloalkyl, alkenyl, and substituted alkenyl;
n is an integer from 1 to 10; and
R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered optionally substituted heterocyclic ring,
with the proviso that if A is

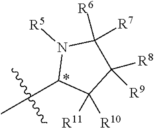

and R⁵ is methyl, then R¹, R², R³, R⁴, R⁶, R⁷, R⁸, R⁹, R¹⁰, and R¹¹ cannot all be H; and
wherein the impregnated toothpick is entirely devoid of (R) or (S) nicotine.

In various embodiments, the an alkaloid dispensing pouch further comprises an oil.

In various examples, the alkaloid composition comprises a substituted pyridine compound of Formula (I) dissolved, dispersed or solubilized in the oil.

In various embodiments, alkaloid compositions used to form impregnated toothpicks in accordance with the present disclosure further comprise at least one organic acid, such that the molar ratio of moles substituted pyridine compound of Formula (I) to moles total organic acid is from about 25:1 to about 75:1. Organic acids for use herein are described below and in the definitions.

In various embodiments, the oil is selected from any partially hydrogenated, or fully hydrogenated vegetable oils such as soybean oil, olive oil, sesame oil, palm oil, coconut oil, and the like. Oils may also be fractionated cuts of fatty acids, fatty acid mixtures, mono-, di-, or triglycerides of fatty acids, wherein the fatty acids and/or glycerides may have a particular chain length distribution (e.g., short-, medium-, or long-chain fatty acids). Of particular use herein are “medium-chain triglycerides” (or “MCT's”), which refers to the glycerin triesters of fatty acids having from about 6 to about 12 carbon atoms, such as caprylic acid, caproic acid, lauric acid, and/or capric acid in any combination, (i.e., C₆-C₁₂ fatty acid triglycerides). In various examples, medium-chain triglycerides can be obtained by fractionating palm kernel oil and coconut oil. Medium-chain triglycerides, USP/NF, CAS No. 73398-61-5, for use in the transdermal THR compositions herein is available, for example, from Specialized Rx Products, LLC, Circle Pines, MN. Also of use herein is Organic MCT Oil-Fractionated Coconut Oil (caprylic/capric triglyceride), CAS Nos. 73398-61-5 and 65381-09-1, having a specific gravity of about 0.930-0.960 g/mL (typically on average, 0.951 g/mL).

In other examples, an oil may comprise a botanical extract, such as cinnamaldehyde, anise oil, peppermint oil, tea tree oil, menthol, benzaldehyde, and so forth. These oils are chosen for the flavor they will impart to a toothpick, and their ability to dissolve, solubilize or disperse the desired substituted pyridine compound. In various embodiments, a substituted pyridine compound of Formula (I) is dissolved in cinnamaldehyde, anise oil, tea tree oil, peppermint oil, or benzaldehyde, etc., and the resulting oily mixture absorbed into wood splints.

Wood splints for alkaloid impregnated toothpicks herein may comprise birch wood or other suitable wood amenable to absorbing an oily mixture.

1g. Impregnated or Coated Dental Floss

In various examples herein, THR dental floss comprises a string or tape impregnated or coated with an alkaloid composition comprising at least one substituted pyridine compound, or salt, or mixed salt thereof, according to Formula (I):

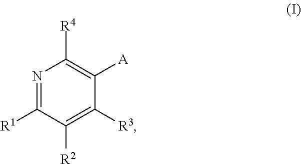

wherein:
A is selected from:

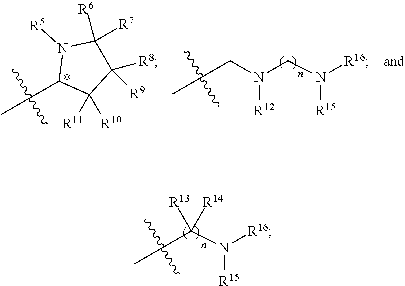

R¹, R², R³, R⁴ and R⁵ are independently selected from H, alkyl, substituted alkyl, cycloalkyl, substituted cycloalkyl, alkenyl, substituted alkenyl, and

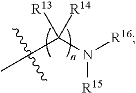

R⁶ and R⁷ are independently selected from H, alkyl, substituted alkyl, cycloalkyl, substituted cycloalkyl, alkenyl and substituted alkenyl, or R⁶ and R⁷ together are ═O;
R⁸, R⁹, R¹⁰, R¹¹, R¹², R¹³, and R¹⁴ are independently selected from H, alkyl, substituted alkyl, cycloalkyl, substituted cycloalkyl, alkenyl, and substituted alkenyl;
n is an integer from 1 to 10; and
R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered optionally substituted heterocyclic ring,
with the proviso that if A is

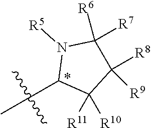

and R⁵ is methyl, then R¹, R², R³, R⁴, R⁶, R⁷, R⁸, R⁹, R¹⁰, and R¹¹ cannot all be H; and
wherein the impregnated or coated dental floss is entirely devoid of (R) or (S) nicotine.

In various embodiments, the an alkaloid dispensing pouch further comprises an oil.

In various examples, the alkaloid composition comprises a substituted pyridine compound of Formula (I) dissolved, dispersed or solubilized in the oil. Depending on the nature of the string or tape, the oily alkaloid composition may coat and/or absorb into the string or tape.

In various embodiments, alkaloid compositions used to form impregnated dental floss in accordance with the present disclosure further comprise at least one organic acid, such that the molar ratio of moles substituted pyridine compound of Formula (I) to moles total organic acid is from about 25:1 to about 75:1. Organic acids for use herein are described below and in the definitions.

In various embodiments, the oil is selected from any partially hydrogenated, or fully hydrogenated vegetable oils such as soybean oil, olive oil, sesame oil, palm oil, coconut oil, and the like. Oils may also be fractionated cuts of fatty acids, fatty acid mixtures, mono-, di-, or triglycerides of fatty acids, wherein the fatty acids and/or glycerides may have a particular chain length distribution (e.g., short-, medium-, or long-chain fatty acids). Of particular use herein are “medium-chain triglycerides” (or “MCT's”), which refers to the glycerin triesters of fatty acids having from about 6 to about 12 carbon atoms, such as caprylic acid, caproic acid, lauric acid, and/or capric acid in any combination, (i.e., C₆-C₁₂ fatty acid triglycerides). In various examples, medium-chain triglycerides can be obtained by fractionating palm kernel oil and coconut oil. Medium-chain triglycerides, USP/NF, CAS No. 73398-61-5, for use in the transdermal THR compositions herein is available, for example, from Specialized Rx Products, LLC, Circle Pines, MN. Also of use herein is Organic MCT Oil-Fractionated Coconut Oil (caprylic/capric triglyceride), CAS Nos. 73398-61-5 and 65381-09-1, having a specific gravity of about 0.930-0.960 g/mL (typically on average, 0.951 g/mL).

In other examples, an oil may comprise a botanical extract, such as cinnamaldehyde, anise oil, peppermint oil, tea tree oil, menthol, and so forth. These oils are chosen for the flavor they will impart to dental floss, and their ability to dissolve, solubilize or disperse the desired substituted pyridine compound. Similar or identical oily alkaloid compositions may be used for both flavored THR toothpicks and flavored THR dental floss.

Dental string or tape for alkaloid impregnated or coated dental floss herein may comprise Nylon or Teflon string or tape. Substrate herein may comprise single or multiple filaments, waxed or unwaxed.

1h. Mouthwash

In various embodiments, a THR mouthwash comprises:
at least one substituted pyridine compound, or salt, or mixed salt thereof, according to Formula (I):

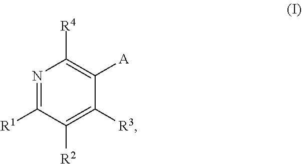

wherein:
A is selected from:

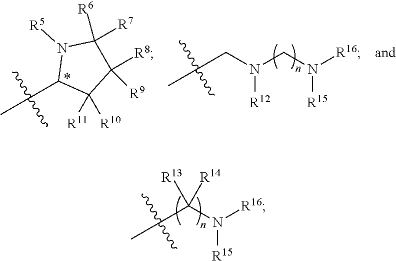

R¹, R², R³, R⁴ and R⁵ are independently selected from H, alkyl, substituted alkyl, cycloalkyl, substituted cycloalkyl, alkenyl, substituted alkenyl, and

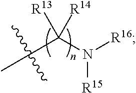

R⁶ and R⁷ are independently selected from H, alkyl, substituted alkyl, cycloalkyl, substituted cycloalkyl, alkenyl and substituted alkenyl, or R⁶ and R⁷ together are ═O;
R⁸, R⁹, R¹⁰, R¹¹, R¹², R¹³, and R¹⁴ are independently selected from H, alkyl, substituted alkyl, cycloalkyl, substituted cycloalkyl, alkenyl, and substituted alkenyl;
n is an integer from 1 to 10; and
R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered optionally substituted heterocyclic ring,
with the proviso that if A is

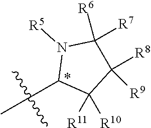

and R⁵ is methyl, then R¹, R², R³, R⁴, R⁶, R⁷, R⁸, R⁹, R¹⁰, and R¹¹ cannot all be H; and
wherein the mouthwash is entirely devoid of (R) or (S) nicotine.

In certain examples, the mouthwash further comprises a diluent.

In certain examples, the diluent is selected from water, alcohol, and mixtures thereof. In some examples, a THR mouthwash comprises from about 10 wt. % to about 30 wt. %, and preferably from about 15 wt. % to about 25 wt. % alcohol, based on the total weight of the mouthwash composition.

In various embodiments, a THR mouthwash can further comprise a foaming surfactant, such as sodium dodecyl sulfate.

In various embodiments, THR mouthwash in accordance with the present disclosure further comprises at least one organic acid, such that the molar ratio of moles substituted pyridine compound of Formula (I) to moles total organic acid is from about 25:1 to about 75:1. Organic acids for use herein are described below and in the definitions.

1i. Gummy Candies

In various embodiments, a THR gummy comprises:
at least one substituted pyridine compound, or salt, or mixed salt thereof, according to Formula (I):

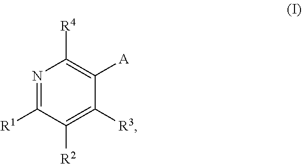

wherein:
A is selected from:

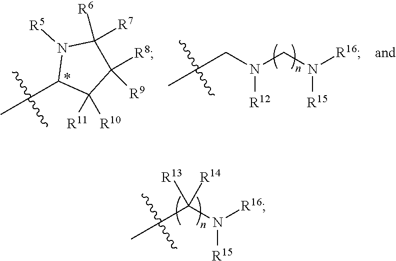

R¹, R², R³, R⁴ and R⁵ are independently selected from H, alkyl, substituted alkyl, cycloalkyl, substituted cycloalkyl, alkenyl, substituted alkenyl, and

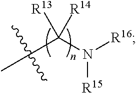

R⁶ and R⁷ are independently selected from H, alkyl, substituted alkyl, cycloalkyl, substituted cycloalkyl, alkenyl and substituted alkenyl, or R⁶ and R⁷ together are ═O;
R⁸, R⁹, R¹⁰, R¹¹, R¹², R¹³, and R¹⁴ are independently selected from H, alkyl, substituted alkyl, cycloalkyl, substituted cycloalkyl, alkenyl, and substituted alkenyl;
n is an integer from 1 to 10; and
R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered optionally substituted heterocyclic ring,
with the proviso that if A is

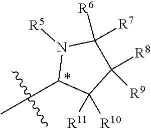

and R⁵ is methyl, then R¹, R², R³, R⁴, R⁶, R⁷, R⁸, R⁹, R¹⁰, and R¹¹ cannot all be H; and
wherein the gummy is entirely devoid of (R) or (S) nicotine.

In certain examples, the gummy further comprises a sugar and/or sugar syrup, a binder, a starch, a wax and combinations thereof

In certain examples, the sugar and/or sugar syrup comprises a mixture of high fructose syrup (e.g., corn syrup) and sucrose. The starch is preferably corn starch.

In various embodiments, the binder is selected from natural gums such as gum Arabic or gum tragacanth, gelatin, pectin, starch, and mixtures thereof, with the preference being gelatin.

Wax is optionally used as a coating or polish on each of the candies and preferably comprises carnauba wax.

In various embodiments, THR gummies in accordance with the present disclosure further comprises at least one organic acid, such that the molar ratio of moles substituted pyridine compound of Formula (I) to moles total organic acid is from about 25:1 to about 75:1. Organic acids for use herein are described below and in the definitions.

1j. Sublingual Disintegrating Tablets

In various embodiments, a THR sublingual tablet comprises:
at least one substituted pyridine compound, or salt, or mixed salt thereof, according to Formula (I):

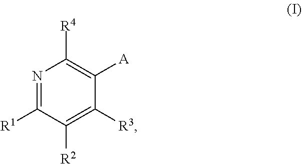

wherein:
A is selected from:

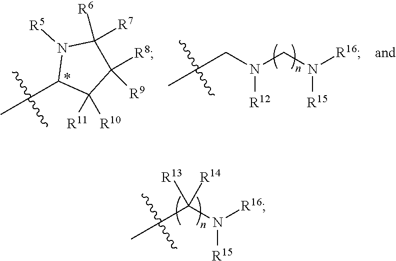

R¹, R², R³, R⁴ and R⁵ are independently selected from H, alkyl, substituted alkyl, cycloalkyl, substituted cycloalkyl, alkenyl, substituted alkenyl, and

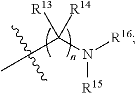

R⁶ and R⁷ are independently selected from H, alkyl, substituted alkyl, cycloalkyl, substituted cycloalkyl, alkenyl and substituted alkenyl, or R⁶ and R⁷ together are ═O;
R⁸, R⁹, R¹⁰, R¹¹, R¹², R¹³, and R¹⁴ are independently selected from H, alkyl, substituted alkyl, cycloalkyl, substituted cycloalkyl, alkenyl, and substituted alkenyl;
n is an integer from 1 to 10; and
R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered optionally substituted heterocyclic ring,
with the proviso that if A is

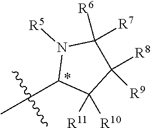

and R⁵ is methyl, then R¹, R², R³, R⁴, R⁶, R⁷, R⁸, R⁹, R¹⁰, and R¹¹ cannot all be H; and
wherein the sublingual tablet is entirely devoid of (R) or (S) nicotine.

In various embodiments, the an alkaloid dispensing pouch further comprises a carrier.

In various embodiments, a THR sublingual tablet composition comprises at least 90 wt. %, at least 95 wt. %, or at least 99 wt. % of a carrier, based on the total weight of the THR sublingual tablet composition. In certain examples, a THR sublingual tablet composition comprises >99.0 wt. %, >99.1 wt. %, >99.2 wt. %, >99.3 wt. %, >99.4 wt. %, >99.5 wt. %, >99.6 wt. %, or >99.7 wt. % of a carrier, based on the total weight of the THR sublingual tablet. A carrier for a THR sublingual tablet composition is a solid substance, or more preferably, a mixture of solid substances that can be in the form of a loose powder.

In certain examples, a carrier for a THR sublingual tablet composition comprises any combination of filler, binder, disintegrant, lubricant, glidant, surfactant, acidic agent, alkaline agent, pH buffering agent, preservative, drug release modifier, coating material, coloring, flavoring, and sweetener, totaling at least 90 wt. %, at least 95 wt. %, or preferably, at least 99 wt. % of the total weight of the THR sublingual tablet composition.

In certain embodiments, a carrier for a THR sublingual tablet composition comprises a filler and a disintegrant. In most cases, the filler represents the majority of the weight of a carrier.

In certain examples, a carrier for a THR sublingual tablet composition comprises a filler, a lubricant, a glidant, and a disintegrant, where the filler represents the majority of the weight of a carrier.

In various embodiments, a carrier for a THR sublingual tablet composition comprises a filler. In certain examples, the filler will comprise the majority of the carrier by wt. %. Fillers include, for example, colloidal anhydrous silica (e.g., Aerosil® 200 or 300 Pharma, or Aeroperl® 300 Pharma), magnesium aluminometasilicate (Neusilin® US2), calcium silicate (Florite®), microcrystalline cellulose powder (e.g., Avicel® PH101 or PH102), silicified microcrystalline cellulose (PROSOLV® SMCC), various starches, and various sugars. Commonly used fillers in pharmaceutical powders and tablets include microcrystalline cellulose (MCC) and/or silicified microcrystalline cellulose (SMCC), and/or a simple sugar, such as mannitol, fructose, or lactose, or combinations of a cellulose and a simple sugar, such as a combination of microcrystalline cellulose and fructose (with either one as the majority wt. %). In various embodiments, a combination of MCC and colloidal silicon dioxide (e.g., 98:2) can substitute for SMCC, although there is some evidence the physical mixture results in some sticking of die punches (see, A. Aljaberi, et al., “Functional performance of silicified microcrystalline cellulose versus microcrystalline cellulose: a case study,” Drug Dev. Ind. Pharma., 35(9), 1066-1071, (2009)).

Other fillers that may find use in THR sublingual tablet compositions of the present disclosure include, for example, cellulose acetate, sorbitol, sucrose, dextrin, dextrose, calcium phosphate dibasic, calcium carbonate, maltose, maltodextrin, kaolin, tribasic calcium phosphate, calcium sulfate, cellulose acetate butyrate (cellaburate), calcium lactate, cellulose acetate, erythritol, ethyl cellulose, ethyl acrylate/methyl methacrylate copolymer, isomalt, α-lactalbumin, lactitol, magnesium carbonate, magnesium oxide, methacrylic acid/ethyl acrylate copolymer, methacrylic acid/methyl methacrylate copolymer, polydextrose, sodium chloride, simethicone, hydrogenated pullulan, talc, amino methacrylate copolymer, trehalose, and xylitol.

In various embodiments, a carrier for a THR sublingual tablet composition comprises a combination of microcrystalline cellulose, colloidal silicon dioxide, mannitol and fructose as a filler, at a level of from about 70 wt. % to about 99 wt. %, based on the total weight of the THR sublingual tablet composition.

In various embodiments, a carrier for a THR sublingual tablet composition comprises a lubricant. Examples of a lubricant include, for example, magnesium stearate, calcium stearate, zinc stearate, sodium lauryl sulfate, sodium stearyl fumarate (SSF), magnesium lauryl sulfate, stearic acid, glyceryl behenate, behenoyl polyoxyl glycerides (e.g., Compritol® HD5), glyceryl dibehenate, lauric acid, glyceryl monostearate, glyceryl tristearate, myristic acid, palmitic acid, poloxamer, polyethylene glycol, polysorbate 20, polyoxyl-10-oleyl ether, polyoxyl-15-hydroxystearate, polysorbate 40, polyoxyl-20-cetostearyl ether, polyoxyl-40-stearate, polysorbate 60, polysorbate 80, potassium benzoate, sodium benzoate, sorbitan monolaurate, sorbitan monooleate, sodium stearate, sorbitan monopalmitate, sorbitan monostearate, zinc stearate, sorbitan sesquioleate, sorbitan trioleate, and talc.

In various embodiments, a carrier for a THR sublingual tablet composition comprises sodium stearyl fumarate (SSF) as a tablet lubricant at a level of from about 0.1 wt. % to about 5 wt. %, based on the total weight of the THR sublingual tablet composition. SSF is available, for example, from JRS Pharma under the trade name PRUV®, or from SPI Pharma under the brand name Lubripharm® SSF.

In various embodiments, a carrier for a THR sublingual tablet composition comprises a binder. Examples of a binder include, for example, polyvinylpyrrolidone (PVP, or povidone), crosslinked polyvinylpyrrolidone (crospovidone) vinylpyrrolidone-vinyl acetate copolymer (copovidone), carbomer, polyethylene glycol (PEG), starches and starch derivatives such as corn starch, pregelatinized starch, carboxymethylcellulose (carmellose), hydroxypropyl methylcellulose (hypromellose), cellulose ethers (e.g., Methocel™), hydroxyethyl cellulose, hydroxypropyl cellulose, hydroxyethyl methyl cellulose, calcium carboxymethylcellulose, calcium cellulose glycolate, ethyl cellulose, chitosan, dextrin, inulin, magnesium aluminum silicate, maltodextrin, methylcellulose, dextrates, sodium alginate, zein, gelatin, polymethacrylates, sorbitol, and acacia.

In various embodiments, a carrier for a THR sublingual tablet composition comprises from none to up to about 20 wt. % binder.

In various embodiments, a carrier for a THR sublingual tablet composition comprises a disintegrant. Examples of a disintegrant include, for example, crospovidone, croscarmellose sodium, sodium starch glycolate, crosslinked sodium carboxymethyl cellulose, low-substituted hydroxpropylcellulose, guar gum, chitosan hydrochloride, calcium alginate, sodium alginate, docusate sodium, magnesium aluminum silicate, methylcellulose, calcium carboxymethylcellulose, and calcium cellulose glycolate.

In various embodiments, a carrier for a THR sublingual tablet composition comprises crospovidone as a disintegrant at a level of from about 0.1 wt. % to about 30 wt. %, based on the total weight of the THR sublingual tablet composition. In certain examples, a THR sublingual tablet composition for tableting comprises from about 2 wt. % to about 5 wt. % crospovidone as the disintegrant, based on the total weight of the THR sublingual tablet composition.

In various embodiments, a carrier for a THR sublingual tablet composition comprises a glidant. Examples of a glidant include, for example, untreated fumed silica (e.g., CAB-O-SIL® M-5P or M-5DP), colloidal silicon dioxide, talc, tribasic calcium phosphate, calcium silicate, magnesium oxide, sodium stearate, magnesium silicate, magnesium trisilicate, and hydrophobic colloidal silica. In preferred embodiments, untreated fumed silica is included in a carrier for a THR sublingual tablet composition as a glidant for improving tablet production.

In various embodiments, a carrier for a THR sublingual tablet composition comprises untreated fumed silica as a glidant at a level of from about 0.5 wt. % to about 1 wt. %, based on the total weight of the THR sublingual tablet composition. The untreated fumed silica acts as a free-flow agent to improve tablet production efficiencies, tablet uniformity, and tablet hardness.

In various embodiments, a carrier for a THR sublingual tablet composition consists essentially of a mixture of untreated fumed silica, sodium stearyl fumarate, microcrystalline cellulose, colloidal silicon dioxide, mannitol, fructose and crospovidone, at a total level of at least 99 wt. %, based on the total weight of the THR sublingual tablet composition.

In various embodiments, a commercially available carrier for use in pharmaceutical tablets may be used as all or part of a carrier in a THR sublingual tablet composition in accordance with the present disclosure. For example, PROSOLV® ODT G2 may be used as the carrier or as the majority of a carrier by weight for THR sublingual tablet compositions for tableting in accordance with the present disclosure. This commercial product, having a bulk density of about 0.45-0.65 g/mL, comprises a mixture of microcrystalline cellulose, colloidal silicon dioxide, mannitol, fructose, and crospovidone, and is available from JRS Pharma, Cedar Rapids, IA.

In various embodiments, a carrier for a THR sublingual tablet composition comprises at least 85 wt. % or at least 90 wt. % of PROSOLV® ODT G2. In certain examples, a carrier for a THR sublingual tablet composition comprises at least 85 wt. %, at least 86 wt. %, at least 87 wt. %, at least 88 wt. %, at least 89 wt. %, or at least 90 wt. % PROSOLV® ODT G2. In various embodiments, a carrier for a THR sublingual tablet composition comprises at least 90 wt. % of PROSOLV® ODT G2 along with about 0.5 wt. % to 1 wt. % untreated fumed silica and about 0.1 wt. % to about 5 wt. % sodium stearyl fumarate. In various formulations, the weight percentage of PROSOLV® ODT G2 may be indicated simply as “quantity sufficient,” or “q.s.” In some examples, the amount of this carrier can be adjusted accordingly to accommodate changes in formulation, such as for example, presence or absence of a flavoring agent or higher or lower alkaloid levels.

A carrier comprising mostly PROSOLV® ODT G2 by weight may also include other separately added fillers, binders, lubricants, disintegrants, and/or glidants as necessary to optimize tablet processing and tablet properties.

In various embodiments, THR sublingual tablets in accordance with the present disclosure further comprise at least one organic acid, such that the molar ratio of moles substituted pyridine compound of Formula (I) to moles total organic acid is from about 25:1 to about 75:1. Organic acids for use herein are described below and in the definitions.

1k. Topical Transdermal Cream

In various embodiments, a THR topical/transdermal cream comprises:
at least one substituted pyridine compound, or salt, or mixed salt thereof, according to Formula (I):

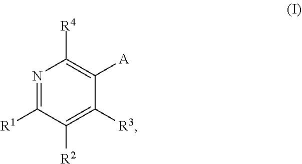

wherein:
A is selected from:

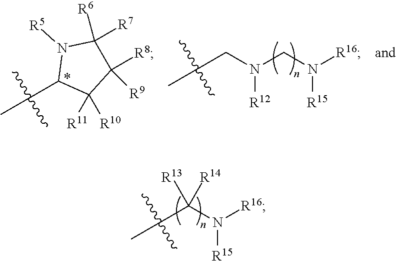

R¹, R², R³, R⁴ and R⁵ are independently selected from H, alkyl, substituted alkyl, cycloalkyl, substituted cycloalkyl, alkenyl, substituted alkenyl, and

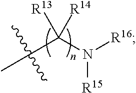

R⁶ and R⁷ are independently selected from H, alkyl, substituted alkyl, cycloalkyl, substituted cycloalkyl, alkenyl and substituted alkenyl, or R⁶ and R⁷ together are ═O;
R⁸, R⁹, R¹⁰, R¹¹, R¹², R¹³, and R¹⁴ are independently selected from H, alkyl, substituted alkyl, cycloalkyl, substituted cycloalkyl, alkenyl, and substituted alkenyl;
n is an integer from 1 to 10; and
R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered optionally substituted heterocyclic ring,
with the proviso that if A is

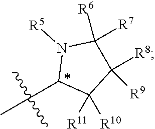

and R⁵ is methyl, then R¹, R², R³, R⁴, R⁶, R⁷, R⁸, R⁹, R¹⁰, and R¹¹ cannot all be H; and
wherein the topical/transdermal cream is entirely devoid of (R) or (S) nicotine.

In various embodiments, the THR topical cream further comprises a solubilizer, a humectant and combinations thereof.

In various embodiments, THR topical cream in accordance with the present disclosure further comprises at least one organic acid, such that the molar ratio of moles substituted pyridine compound of Formula (I) to moles total organic acid is from about 25:1 to about 75:1. Organic acids for use herein are described below and in the definitions.

The solubilizer is selected from partially hydrogenated, or fully hydrogenated vegetable oils such as soybean oil, olive oil, sesame oil, palm oil, coconut oil, and the like. Solubilizers may also be fractionated cuts of fatty acids, fatty acid mixtures, mono-, di-, or triglycerides of fatty acids, wherein the fatty acids and/or glycerides may have a particular chain length distribution (e.g., short-, medium-, or long-chain fatty acids). Of particular use herein are “medium-chain triglycerides” (or “MCT's”), which refers to the glycerin triesters of fatty acids having from about 6 to about 12 carbon atoms, such as caprylic acid, caproic acid, lauric acid, and/or capric acid in any combination, (i.e., C₆-C₁₂ fatty acid triglycerides). In various examples, medium-chain triglycerides can be obtained by fractionating palm kernel oil and coconut oil. Medium-chain triglycerides, USP/NF, CAS No. 73398-61-5, for use in the transdermal THR compositions herein is available, for example, from Specialized Rx Products, LLC, Circle Pines, MN. Also of use herein is Organic MCT Oil-Fractionated Coconut Oil (caprylic/capric triglyceride), CAS Nos. 73398-61-5 and 65381-09-1, having a specific gravity of about 0.930-0.960 g/mL (typically on average, 0.951 g/mL)

A solubilizer for use in various THR topical creams may also comprise a terpene, such as geraniol, citronellol, geranial, citronellal, linalool, menthone, rose oxide, α-terpineol and limonene.

Humectants for use herein include glycerin, honey, 1,2-propylene glycol, 1,3-propylene glycol, various carboxylic acid esters of terpene alcohols, lecithin, lanolin, triethylene glycol, xylitol, sorbitol, hexylene glycol, and urea.

In various embodiments, a THR topical cream further comprises a chemosensory irritant, such as to impart a cooling sensation or for pain relief. In particular, a cannabinoid may be included in the THR product, and/or menthol and/or capsaicin.

### 1l Ion Exchange Resins

In various embodiments, any of the substituted pyridine compounds, or salts, or mixed salts thereof are bound to an ion-exchange resin.

In various embodiments, the pyridine compound, salt, or mixed salt thereof bound to the ion-exchange resin comprises a pyridine compound, salt, or mixed salt thereof, according to Formula (I):

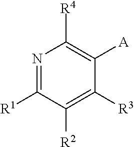

A is selected from:

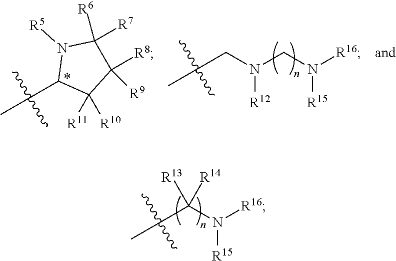

R¹, R², R³, R⁴ and R⁵ are independently selected from H, alkyl, substituted alkyl, cycloalkyl, substituted cycloalkyl, alkenyl, substituted alkenyl, and

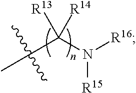

R⁶ and R⁷ are independently selected from H, alkyl, substituted alkyl, cycloalkyl, substituted cycloalkyl, alkenyl and substituted alkenyl, or R⁶ and R⁷ together are ═O;
R⁸, R⁹, R¹⁰, R¹¹, R¹², R¹³, and R¹⁴ are independently selected from H, alkyl, substituted alkyl, cycloalkyl, substituted cycloalkyl, alkenyl, and substituted alkenyl;
n is an integer from 1 to 10; and
R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered optionally substituted heterocyclic ring,

With the proviso that if A is

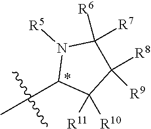

and R⁵ is methyl, then R¹, R², R³, R⁴, R⁶, R⁷, R⁸, R⁹, R¹⁰, and R¹¹ cannot all be H; and
wherein the compound is entirely devoid of (R) or (S) nicotine.

In some embodiments, the substituted pyridine compounds, or salts, or mixed salts thereof further comprise a carrier.

In some embodiments, the pyridine compound is a derivative of a (S)-6-methyl nicotine compound. In some embodiments, the derivate of the (S)-6-methyl nicotine compounds comprises Formula (I):

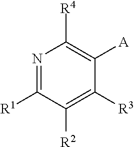

wherein A comprises:

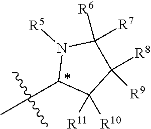

In some embodiments, any of the pyridine compounds, salts, or mixed salts thereof or (S)-6 methyl nicotine derivatives are bound to a carrier. In some embodiments, the carrier comprises an ion-exchange resin. In some embodiments, the ion-exchange resin is configured to deliver and release any of the pyridine or (S)-6 methyl nicotine products described herein to the oral mucosa. In some embodiments, the ion-exchange resin facilitates absorption of any of the pyridine or (S)-6 methyl nicotine products described herein by the oral mucosa. In some embodiments, the ion-exchange resin results in uniform release of the pyridine compound or the (S)-6-methyl nicotine derivative.

In some embodiments, the ion-exchange resin comprises an acid. In some embodiments, the ion-exchange resin comprises a strong acid. In some embodiments, the ion-exchange resin comprises a weak acid. In some embodiments, the ion-exchange resin comprises a base. In some embodiments, the ion-exchange resin comprises a strong base. In some embodiments, the ion-exchange resin comprises a weak base.

In some embodiments, the ion-exchange resin comprises an anion-exchange resin. In some embodiments, the anion-exchange resin comprises a functional group. In some embodiments, the functional group comprises an amine group. In some embodiments, the functional group comprises quaternary ammonium. In some embodiments, the functional group comprises ammonium chloride. In some embodiments, the functional group comprises hydroxide.

In some embodiments, the anion-exchange resin comprises a copolymer. In some embodiments, the copolymer comprises styrene. In some embodiments, the copolymer comprises divinylbenzene.

In some embodiments, the ion-exchange resin comprises a cation-exchange resin. In some embodiments, the cation-exchange resin comprises a functional group. In some embodiments, the functional group comprises sulfonic acid. In some embodiments, the functional group comprises carboxylic acid.

In some embodiments, the ion-exchange resin comprises a cation-exchange resin. In some embodiments, the cation-exchange resin comprises a polystyrene polymer. In some embodiments, the cation-exchange resin comprises sodium polystyrene sulfonate. In some embodiments, the cation-exchange resin comprises sodium zirconium cyclosilicate. In some embodiments, the cation exchange resin comprises sodium polystyrene sulfonate.

In various embodiments, the ion-exchange resin comprises an acrylic polymer. In some embodiments, the acrylic polymer comprises derivatives of acrylic acid. In some embodiments, the acrylic polymer comprises derivatives of methylacrylic acid. In some embodiments, the acrylic polymer comprises derivatives of acrylic acid and methyl acrylic acid.

In some embodiments, the ion-exchange resin comprises a copolymer. In some embodiments, the copolymer comprises an acrylic polymer. In some embodiments, the acrylic polymer comprises derivatives of acrylic acid. In some embodiments, the acrylic polymer comprises derivatives of methylacrylic acid. In some embodiments, the acrylic polymer comprises derivatives of acrylic acid and methyl acrylic acid. In some embodiments, the copolymer comprises a vinylic monomer. In some embodiments, the copolymer comprises an allylic monomer.

In some embodiments, the resin comprises poly(methacrylic acid) (PMAA) (poly(1-methylprop-1-enoic acid). In some embodiments, PMAA comprises a formula of (C₄H₆O₂)ₙ.

In some embodiments, the ion-exchange resin comprises a copolymer comprising poly(methacrylic acid) that is cross-linked with another compound.

In some embodiments, the ion-exchange resin comprises a copolymer comprising poly(methacrylic) acid and divinylbenzene. In some embodiments, any of the pyridine compounds or (S)-6-methyl nicotine derivatives is bound to an ion-exchange resin comprising poly(methacrylic) acid.

In various embodiments, the copolymer comprising poly(methacrylic) acid and divinylbenzene comprises Amberlite IRP64 or a derivative thereof. In some embodiments, any of the pyridine compounds or (S)-6-methyl nicotine derivatives is bound to an ion-exchange resin comprising Amberlite IPR64 or a derivative thereof.

In some embodiments, the copolymer comprising poly(methacrylic) acid and divinylbenzene comprises Purolite C115HMR or a derivative thereof. In some embodiments, any of the pyridine compounds or (S)-6-methyl nicotine derivatives is bound to an ion-exchange resin comprising Purolite C115HMR or a derivative thereof.

In some embodiments, the copolymer comprising poly(methacrylic) acid and divinylbenzene comprises Doshion P551. In some embodiments, any of the pyridine compounds or (S)-6-methyl nicotine derivatives is bound to a ion-exchange resin comprising Doshion P551 or a derivative thereof.

In various embodiments, any of the pyridine compounds, salts, or mixed salts thereof bound to an ion-exchange resin are added to a gum to be used as part of a smoking cessation therapy. In some embodiments, the gum comprises a candy coated gum. In some embodiments, the gum comprises small beads. In some embodiments, the gum comprises flat sticks of gum. In some embodiments, the gum comprises bubble gum. In some embodiments, the gum comprises shredded bubble gum. In some embodiments, any of the pyridine compounds, salts, or mixed salts thereof, or (S)-6-methyl nicotine derivatives bound to an ion-exchange resin are added to any gum described herein.

In various embodiments, any of the pyridine compounds, salts, or mixed salts thereof bound to an ion-exchange resin are added to a hard lozenge to be used as part of a smoking cessation therapy. In some embodiments, the lozenge comprises a dissolvable lozenge. In some embodiments, the lozenge comprises a lozenge that dissolves slowly in the mouth of an individual, such as over the course of several minutes. In some embodiments, any of the pyridine compounds, salts, or mixed salts thereof, or (S)-6-methyl nicotine derivatives bound to an ion-exchange resin are added to any hard lozenge described herein.

### 1m Salt Forms of (S)-6-Methyl Nicotine Derivatives

In various embodiments, (S)-6-methyl nicotine or derivatives thereof are disclosed herein. In some embodiments, the (S)-6-methyl nicotine or derivatives thereof comprise a pyridine compound.

In various embodiments, the (S)-6-methyl nicotine structure or derivative thereof comprises a compound according to Formula (I):

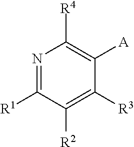

In embodiments, A comprises the structure:

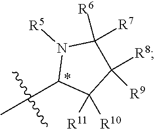

R¹, R², R³, R⁴ and R⁵ are independently selected from H, alkyl, substituted alkyl, cycloalkyl, substituted cycloalkyl, alkenyl, substituted alkenyl, and

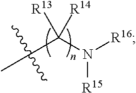

R⁶ and R⁷ are independently selected from H, alkyl, substituted alkyl, cycloalkyl, substituted cycloalkyl, alkenyl and substituted alkenyl, or R⁶ and R⁷ together are ═O;
R⁸, R⁹, R¹⁰, R¹¹, R¹², R¹³, and R¹⁴ are independently selected from H, alkyl, substituted alkyl, cycloalkyl, substituted cycloalkyl, alkenyl, and substituted alkenyl;
n is an integer from 1 to 10; and
R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered optionally substituted heterocyclic ring,

With the proviso that if R⁵ is methyl, then R¹, R², R³, R⁴, R⁶, R⁷, R⁸, R⁹, R¹⁰, and R¹¹ cannot all be H; and
wherein the compound is entirely devoid of (R) or (S) nicotine.

In various embodiments, any of the (S)-6 methyl nicotine or derivates thereof comprises a carrier.

In various embodiments, any of the (S)-6 methyl nicotine or derivates thereof comprises a pharmaceutically acceptable salt. In some embodiments, the pharmaceutically acceptable salt comprises a hydrogen tartrate salt.

In some embodiments, the hydrogen tartrate salt comprises potassium hydrate tartrate. In some embodiments, the potassium hydrate tartrate comprises the formula KC₄H₅O₆.

In some embodiments, the pharmaceutically acceptable salt comprises bitartrate dihydrate or a derivative thereof. In some embodiments, the bitartrate dihydrate comprises the formula of 2C₄H₆O₆, 2H₂O. In some embodiments, the bitartrate dihydrate comprises a derivative of the formula of 2C₄H₆O₆, 2H₂O.

In some embodiments, the pharmaceutically acceptable salt comprises any of the (S)-6 methyl nicotine compounds or derivatives thereof described herein, and a compound of the formula of 2C₄H₆O₆, 2H₂O or a derivative thereof.

In some embodiments, the pharmaceutically acceptable salt comprises at least one water molecule. In some embodiments, the pharmaceutically acceptable salt comprises at least two water molecules, for example, a dihydrate. In some embodiments, the pharmaceutically acceptable salt comprises more than two water molecules, for example, at least three water molecules, at least four water molecules, at least five water molecules, at least six water molecules, at least seven water molecules, at least eight water molecules, at least nine water molecules, or at least ten water molecules.

In various embodiments, the pharmaceutically acceptable salt comprises any of the (S)-6-methyl nicotine structures or derivatives and tartrate. In some embodiments, tartrate comprises the formula of C₄H₄O₆²⁻. In some embodiments, tartrate comprises a derivative of C₄H₄O₆²⁻. In some embodiments, the tartrate comprises a sodium tartrate. In some embodiments, the sodium tartrate comprises any one or more of monosodium tartrate, sodium tartrate, and sodium ammonium tartrate. In some embodiments, the tartrate comprises potassium tartrate. In some embodiments, the potassium tartrate comprises any one or more of potassium bitartrate, potassium tartrate, potassium sodium tartrate, calcium tartrate, and stearyl tartrate.

In some embodiments, the pharmaceutically acceptable salt comprises a bitartrate salt or bitartrate anion (3-Carboxy-2,3-dihydroxypropanoate). In some embodiments, the bitartrate salt comprises choline bitartrate. In some embodiments, the bitartrate salt comprises potassium bitartrate. In some embodiments, the bitartrate sale comprises sodium bitartrate.

In some embodiments, any of the pharmaceutically acceptable salts of the (S)-6 methyl nicotine derivatives described herein are added to a gum to be used as part of a smoking cessation therapy. In some embodiments, the gum comprises a candy coated gum. In some embodiments, the gum comprises small beads. In some embodiments, the gum comprises flat sticks of gum. In some embodiments, the gum comprises bubble gum. In some embodiments, the gum comprises shredded bubble gum. In some embodiments, any of the pharmaceutically acceptable salts of the (S)-6 methyl nicotine derivatives described herein are added to any gum described herein.

In various embodiments, any of the pharmaceutically acceptable salts of the (S)-6 methyl nicotine derivatives described herein are added to a hard lozenge to be used as part of a smoking cessation therapy. In some embodiments, the lozenge comprises a dissolvable lozenge. In some embodiments, the lozenge comprises a lozenge that dissolves slowly in the mouth of an individual, such as over the course of several minutes. In some embodiments, any of the pharmaceutically acceptable salts of the (S)-6 methyl nicotine derivatives described herein are added to any hard lozenge described herein.

### Other Ingredients

In various embodiments, THR products, including the subset of THR pain relief products, may further comprise any combination of flavoring agent, sweetener, colorant, disintegrant, permeation enhancer, stabilizer, preservative, or chemosensory irritant. Any of these materials not specifically mentioned herein may be found in “Handbook of Pharmaceutical Excipients,” 6^{th} Edition, R. C Rowe, et al., editors, Pharmaceutical Press, London, 2009.

Suitable flavoring agents can include, for example, natural flavors, artificial flavors, and combinations thereof. Non-limiting examples of flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Suitable flavoring agents also include, for example, artificial, natural and synthetic fruit flavors such as vanilla, citrus oils (e.g., lemon, orange, lime, and grapefruit), and fruit essences (e.g., apple, pear, peach, grape, strawberry, raspberry, cherry, plum, pineapple, and apricot), and the like, and combinations thereof.

Other flavoring agents and fragrant aromatics that may be included individually or in combination include, but are not limited to, anethole, menthol, menthone, menthyl acetate, eucalyptol, borneol, borneol acetate, camphor, 1,8-cineole, cinnamaldehyde, benzaldehyde, citral, thujone, eugenol, limonene, geraniol, citronellol, citronellal, pinene, linalool, thymol, carvone, caryophyllene, linalyl acetate, methyl salicylate, and mixtures thereof. Also, substances that provide scent and flavor include, but are not limited to, 3,3,5-trimethylcyclohexanol, methoxycyclohexanol, benzyl alcohol, anise alcohol, cinnamyl alcohol, β-phenyl ethyl alcohol (2-phenylethanol), cis-3-hexenol, musk xylol, isoeugenol, methyl eugenol, α-amylcinnamic aldehyde, anisaldehyde, n-butyl aldehyde, cumin aldehyde, cyclamen aldehyde, decanal, isobutyl aldehyde, hexyl aldehyde, heptyl aldehyde, n-nonyl aldehyde, nonadienol, hydroxycitronellal, benzaldehyde, methyl nonyl acetaldehyde, dodecanol, α-hexylcinnamic aldehyde, undecenal, heliotropin, vanillin, ethyl vanillin, methyl amyl ketone, methyl β-naphthyl ketone, methyl nonyl ketone, musk ketone, diacetyl, acetyl propionyl, acetyl butyryl, acetophenone, p-methyl acetophenone, ionone, methyl ionone, amyl butyrolactone, diphenyl oxide, methyl phenyl glycidate, γ-nonyl lactone, coumarin, cineole, ethyl methyl phenyl glycidate, methyl formate, isopropyl formate, linalyl formate, ethyl acetate, octyl acetate, methyl acetate, benzyl acetate, butyl propionate, isoamyl acetate, isopropyl isobutyrate, geranyl isovalerate, allyl capronate, butyl heptylate, octyl caprylate octyl, methyl heptynecarboxylate, methine octynecarboxylate, isoacyl caprylate, methyl laurate, ethyl myristate, methyl myristate, ethyl benzoate, benzyl benzoate, methylcarbinylphenyl acetate, isobutyl phenylacetate, methyl cinnamate, cinnamyl cinnamate, ethyl anisate, methyl anthranilate, ethyl pyruvate, ethyl α-butyl butylate, benzyl propionate, butyl acetate, butyl butyrate, p-tert-butylcyclohexyl acetate, cedryl acetate, citronellyl acetate, citronellyl formate, p-cresyl acetate, ethyl butyrate, ethyl caproate, ethyl cinnamate, ethyl phenylacetate, ethylene brassylate, geranyl acetate, geranyl formate, isoamyl salicylate, isoamyl isovalerate, isobornyl acetate, linalyl acetate, methyl anthranilate, methyl dihydrojasmonate, β-phenylethyl acetate, trichloromethylphenyl carbinyl acetate, terpinyl acetate, vetiveryl acetate, and mixtures thereof.

Suitable sweeteners include nutritive carbohydrates such as sucrose, glucose, fructose, glucose, trehalose, galactose, mannitol, sorbitol, and xylitol, as well as artificial sweeteners such as saccharin, aspartame, acesulfame K, cyclamates, neotame, sucralose, and neohesperidin dihydrochalcone (NHDC) and the naturally obtainable, non-sugar sweetener, stevia (i.e., steviol glycosides obtained from the plant species, Stevia rebaudiana.

Exemplary colorants for use in THR products include pharmaceutically acceptable colorants such as the U.S. FDA certified colors, dyes and lakes seen in pharmaceutical capsules, tablets and syrups. These acceptable colorants include the inorganic pigments such as titanium dioxide, yellow iron oxide, red iron oxide and black iron oxide, the organic pigments such as D&C Red 36, Red 30 and Red 34, the solvent soluble colors D&C Yellow 11, Yellow 7, Red 27, Red 21, Red 17, Green 6, and Violet 2, and the water soluble colors D&C Green 8, Yellow 10, Yellow 8, Orange 4, Red 22, Red 28, Red 33, Green 5, quinoline yellow, FD&C Yellow 5, Yellow 6, Red 4, Red 40, Red 3, Green 3, Blue 1, Blue 2, and ponceau 4R, carmoisine, amaranth, patent blue V and black PN, and a number of “organic lakes.”

Oral THR products designed to dissolve quickly, such as sublingual THR tablets and THR dissolving oral strips, may include a disintegrant. Suitable disintegrants include, but are not limited to, sodium starch glycolate, croscarmellose sodium, microcrystalline cellulose, and crospovidone. For a review of disintegrants that find use in various THR products herein, see P. M. Desai, “Review of Disintegrants and the Disintegration Phenomena,” J. Pharm. Sci., 105, 2545-2555 (2016).

THR products herein, such as a THR lozenge, may further comprise an intestinal permeation enhancer, distinguishable from transdermal permeation enhancers that find use in the pain relief THR products herein. Suitable intestinal permeation enhancers include, but are not limited to, surfactants that assist bio-absorption, including, for example, fatty acids and/or esters or salts thereof, bile acids and/or salts thereof. Bile acids/salts and fatty acids and their uses are further described in U.S. Pat. No. 6,287,860, which is incorporated herein in its entirety. In some embodiments of oral THR products, the present disclosure provides combinations of absorption enhancers, for example, fatty acids/salts in combination with bile acids/salts. An exemplary combination is the sodium salt of lauric acid, capric acid and ursodeoxycholic acid (UDCA) for promoting improved intestinal absorption of peptides and other materials. These excipients may be used in the present THR compositions to assist absorption of the substituted pyridine compound, salt or mixed salt thereof. Further penetration enhancers include, but are not limited to, polyoxyethylene-9-lauryl ether, polyoxyethylene-20-cetyl ether. For a review of absorption enhancers that find use herein, see B. J. Aungst, “Intestinal Permeation Enhancers,” J. Pharm. Sci., 89(4), 429 (2000).

In various embodiments, THR products herein may further comprise a stabilizer or preservative. Such substances for oral products include the parabens, sorbitol, sodium benzoate, benzoic acid, sorbic acid, potassium sorbate, propionic acid, and combinations thereof. Antioxidants include, but are not limited to, vitamin C, vitamin E, butylated hydroxyanisole (BHA), butylated hydroxytoluene (BHT), and propyl gallate. For a review see, I. Himoudy, “Preservatives and their role in pharma and clinical research,” International Journal of Pharma Sciences and Scientific Research, 2:4, 134-151 (2016).

Inclusion of a chemosensory irritant in various THR products of the present disclosure is optional. When used in the compositions herein, a chemosensory irritant includes any natural, endogenous, phytochemical, or synthetic compound, including those derived from naturally occurring compounds by organic chemistry, capable of activating a TRP ion channel. Assays usable to determine whether a compound is indeed a TRP ion channel activator are found, for example, in H. P. Fallah, et al., “A Review on the Role of TRP Channels and Their Potential as Drug Targets: An Insight Into the TRP Channel Drug Discovery Methodologies,” Front. Pharmacol., 2022 13:914499.

Chemosensory irritants for use herein include, but are not limited to, oleocanthal, 4-hydroxy-2-nonenal, 4-oxo-2-nonenal, allicin, allyl isothiocyanate, icilin, polygodial, cinnamaldehyde, trans-p-methoxycinnamaldehyde, methyl syringate, 2-chlorobenzylidene malononitrile, 1-chloroacetophenone, ethyl bromoacetate, 4-hydroxyhexenal, toluene diisocyanate, p-benzoquinone, methyl p-hydroxybenzoate, flufenamic acid, niflumic acid, mefenamic acid, diclofenac, hydroxy-α-sanshool, 6-paradol, linalool, carvacrol, eugenol, thymol, vanillin, methyl eugenol, 2,6-dimethylphenol, 2,5-dimethylphenol, 3,4-dimethylphenol, 2,6-diisopropylphenol, caffeine, farnesyl thiosalicylic acid, 4-allylanisole, curcumin, niacin, niacinamide, camphor, olvanil, arvanil, anandamide (AEA), 2-AG, NADA, OLDA, PEA, NGABA, NGly, NAsp, NSer, Δ⁹-tetrahydrocannabinol (Δ⁹-THC), Δ⁹-THCA, Δ⁹-THCV, Δ⁹-THCVA, CBD, CBDA, CBDV, CBG, CBGA, CBGV, CBN, CBC, WIN55, 212-2, AM630, (R)-AM1241, (S)-AM1241, SR141716A, Gp-1a, AM251, SR144528, JWH133, HU308, HU910, CP55,940, Nabilone, various phytochemicals from Euphorbia reinfera, Euenia carophyllata, Ocimum gratissiumum, Panax, Aframomum melgueta, Evodia rutaecarpa, Drymis winteri, Cinnamosma fragrans, Warburgia, Scutellaria baicalensis, Vitex agnus, Pterodon pubescens, Croton macrostachyus, Angelicae pubescentis, Ephedra sinica, Amphilophium crucigerum, Bosewellia carterii, Commiphora myrrha, Echinophora platyloba, Nypa fruticans, Corydalis saxicola, Coptis chinensis, Ononis spinosa, Parkia platycephala, and Cymbopogon citratus, and the entirety of a subset of chemosensory irritants separately defined as spice additives having a spiciness of from about 1,000 SHU to about 20,000,000 SHU. Many spice additives for use herein are phytochemicals found in various herbs and fruits, such as compounds from Capsicum annuum, Zingiber officiale, and Piper nigrum.

In accordance with various embodiments, a spice additive for use as a chemosensory irritant in the various THR products of the present disclosure is an organic compound having a spiciness or heat of about 1,000 to about 20,000,000 Scoville Heat Units (SHU), and more preferably from about 100,000 to about 20,000,000 Scoville Heat Units (SHU). Thus, a spice additive for purposes herein is a defined group of chemical compounds. Calculation of Scoville Heat Units can be found in American Spice Trade Associate (ASTA) 1985 Official Analytical Methods of the American Spice Trade Association, 3^{rd} ed., Amer. Spice Trade Assn., Englewood Cliffs, N.J.

In various embodiments, a spice additive herein comprises a capsaicinoid or mixture of capsaicinoids. In certain examples, a liquid extract comprises a mixture of capsaicinoids, having been prepared by solvent extraction of chili peppers known to contain mixtures of capsaicinoids rather than only capsaicin. In other embodiments, capsaicin is sourced and used.

In other examples, spice additives are used that are not chemically related to capsaicinoids, but nonetheless have measurable heat levels reported in Scoville units. These compounds are typically extracted from peppers other than chili peppers (Capsicum annuum), such as black pepper (Piper nigrum L.), with the most useful spice additive that is not a capsaicinoid being piperine (E, E, or trans-trans isomer). Other spice additives for use herein that are not capsaicinoids include, but are not limited to, Shogaol, gingerol, isopiperine, chavicine, isochavicine, 2-piperamine, piperanine (4,5-dihydropiperine), piperamide, 4-pipericide, piperyline, piperlonguminine, piperettine, piperdardine (6,7-dihydropiperettine), 5-sarmentodine, 6-sarmentine, and 7-trichostachine.

In various embodiments, a spice additive is selected from the group consisting of capsaicin, dihydrocapsaicin, norcapsaicin, nordihydrocapsaicin, homocapsaicin, homodihydrocapsaicin, gingerol, piperine, Shogaol, and mixtures thereof. Other capsaicinoids besides those six articulated may be used in the compositions herein, noting that it has been described that there are at least seven different capsaicinoids in the genus Capsicum annuum and perhaps two of them, likely capsaicin and dihydrocapsaicin, together are the capsaicinoids responsible for the spiciness of chili peppers. See M. D. Collins, et al., “Improved Method for Quantifying Capsaicinoids in Capsicum Using High-Performance Liquid Chromatography,” HortScience 30(1):137-139, 1995. Although the above-referenced compounds fall in the general class of chemosensory irritants, it is convenient to group these compounds in the subset of chemosensory irritants defined as spice additives having a spiciness of from about 1,000 to about 20,000,000 Scoville Heat Units (SHU).

In certain embodiments, a THR product may comprise capsaicin ((E)-N-[(4-hydroxy-3-methoxyphenyl)methyl]-8-methylnon-6-enamide, CAS No.: 404-86-4). In a pure form (100% active), capsaicin is a white crystalline solid with a MP of 65° C. The purified material may be used directly in the products herein and is available, for example, from Sigma-Aldrich Co., VWR Avantor, and Alfa Chemistry, amongst other chemical suppliers.

In various embodiments, a THR product herein comprises from 0 wt. % to about 0.01 wt. % capsaicin ((E)-N-[(4-hydroxy-3-methoxyphenyl)methyl]-8-methylnon-6-enamide), based on the total weight of the alkaloid composition. In other examples, the amount of capsaicin is from 0 wt. % to about 0.0015 wt. %, based on the total weight of the alkaloid composition used in the THR product.

In various example, a “liquid capsaicin” is used such as for cost and practicality reasons. For example, Capsicum Flavor Extract can be used, which is available in gallon up to drum quantities from OliveNation, LLC, Avon, MA. This product is an ethanolic extract of chili peppers, having a clear reddish brown appearance and a specific gravity of about 0.8 g/mL at 20° C. This commercial material is about 99.1 wt. % ethanol and about 0.9 wt. % capsaicinoid actives.

### Further Aspects of the Substituted Pyridine Compounds of Formula (I)

With reference to Formula (I), and independent of all other variables, A is:

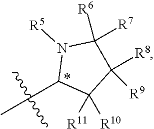

and R⁵ is selected from —(CH₂)—NH₂; —(CH₂)₂—NH₂; —(CH₂)₃—NH₂; —(CH₂)₄—NH₂; —(CH₂)₅—NH₂; —(CH₂)₆—NH₂; —(CH₂)₇—NH₂; —(CH₂)₈—NH₂; —(CH₂)₉—NH₂; —(CH₂)₁₀—NH₂; —(CH₂)—N(CH₃)₂; —(CH₂)₂—N(CH₃)₂; —(CH₂)₃—N(CH₃)₂; —(CH₂)₄—N(CH₃)₂; —(CH₂)₅—N(CH₃)₂; —(CH₂)₆—N(CH₃)₂; —(CH₂)₇—N(CH₃)₂; —(CH₂)₈—N(CH₃)₂; —(CH₂)₉—N(CH₃)₂; and —(CH₂)₁₀—N(CH₃)₂.

In various embodiments, and independent of all other variables, A is:

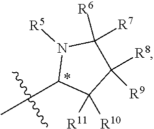

and R⁵ is selected from:

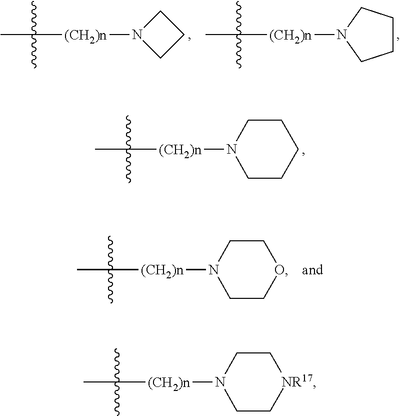

wherein n is 1-10; and R¹⁷ is selected from H, alkyl, substituted alkyl, cycloalkyl, substituted cycloalkyl, alkenyl, and substituted alkenyl.

In various embodiments, and independent of all other variables, A is:

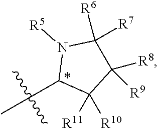

### and R⁵ is

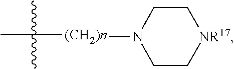

wherein n is 1-10; and R¹⁷ is H or CH₃.

In various embodiments, a substituted pyridine compound for use herein has a structure according to Formula (IA):

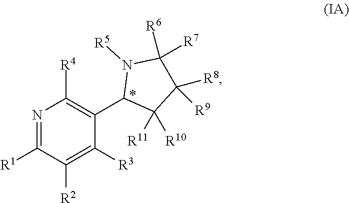

wherein all variables are as previously defined.

In various embodiments, a substituted pyridine compound for use herein has a structure according to Formula (IA-1):

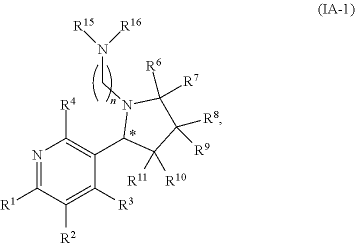

wherein all variables are as previously defined.

In various embodiments, a substituted pyridine compound for use herein has a structure according to Formula (IA-2):

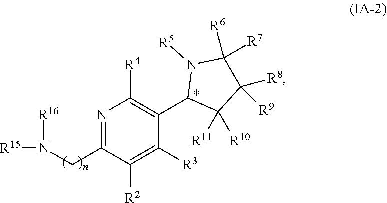

wherein all variables are as previously defined.

In various embodiments, a substituted pyridine compound for use herein has a structure according to Formula (IA-3):

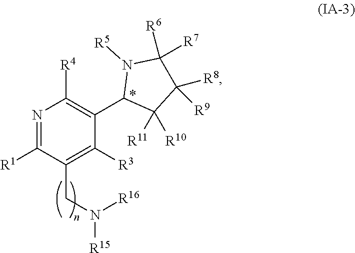

wherein all variables are as previously defined.

In various embodiments, a substituted pyridine compound for use herein has a structure according to Formula (IA-4):

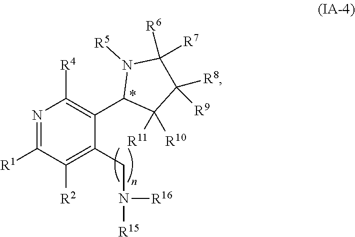

wherein all variables are as previously defined.

In various embodiments, a substituted pyridine compound for use herein has a structure according to Formula (IA-5):

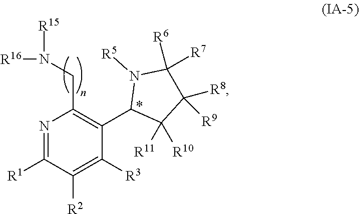

wherein all variables are as previously defined.

In various embodiments, a substituted pyridine compound for use herein has a structure according to Formula (IB):

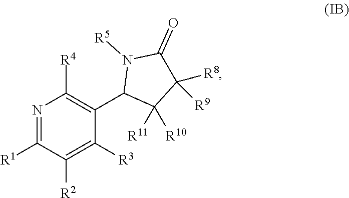

wherein all variables are as previously defined.

In various embodiments, a substituted pyridine compound for use herein has a structure according to Formula (IC):

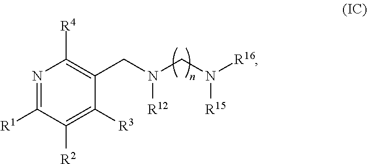

wherein all variables are as previously defined.

In various embodiments, a substituted pyridine compound for use herein has a structure according to Formula (ID):

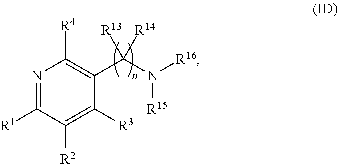

wherein all variables are as previously defined.

In various embodiments, a THR product herein comprises a substituted pyridine compound selected from:

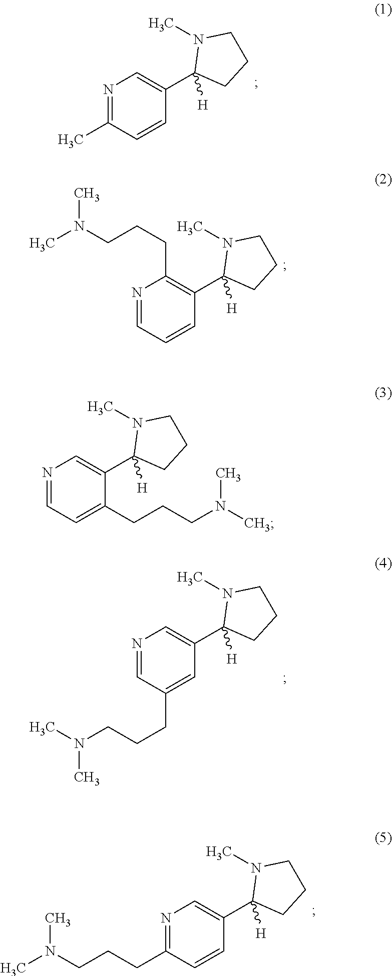 [SMILES: Cc1ccc(C2CCCN2C)cn1 ; CN(C)CCCc1ncccc1C1CCCN1C ; CN(C)CCCc1ccncc1C1CCCN1C ; CN(C)CCCc1cncc(C2CCCN2C)c1 ; CN(C)CCCc1ccc(C2CCCN2C)cn1]

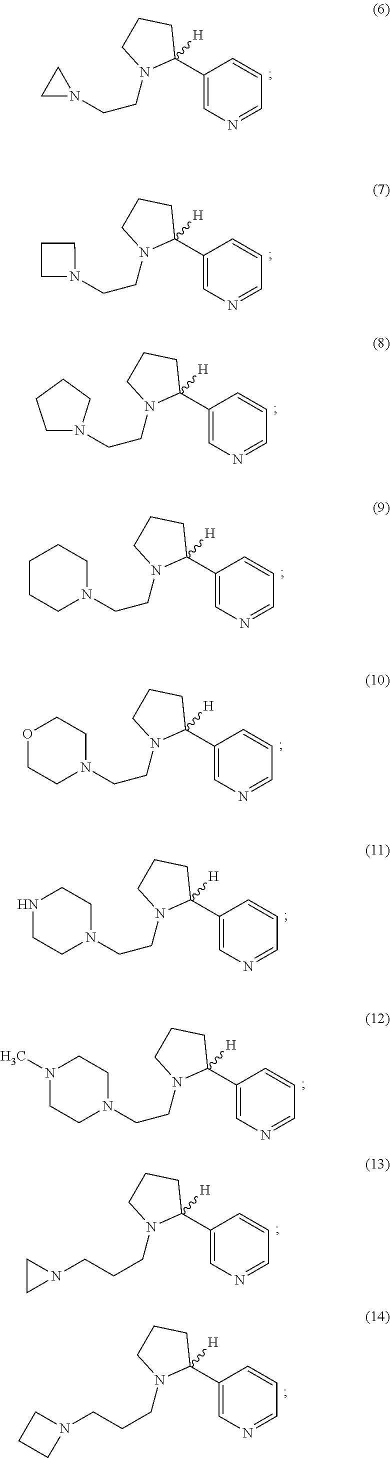

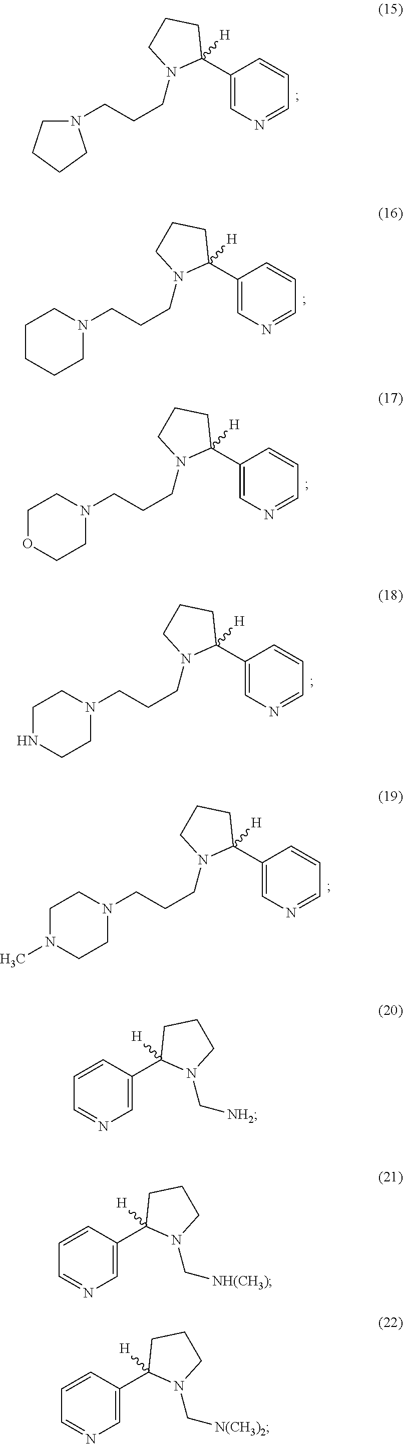

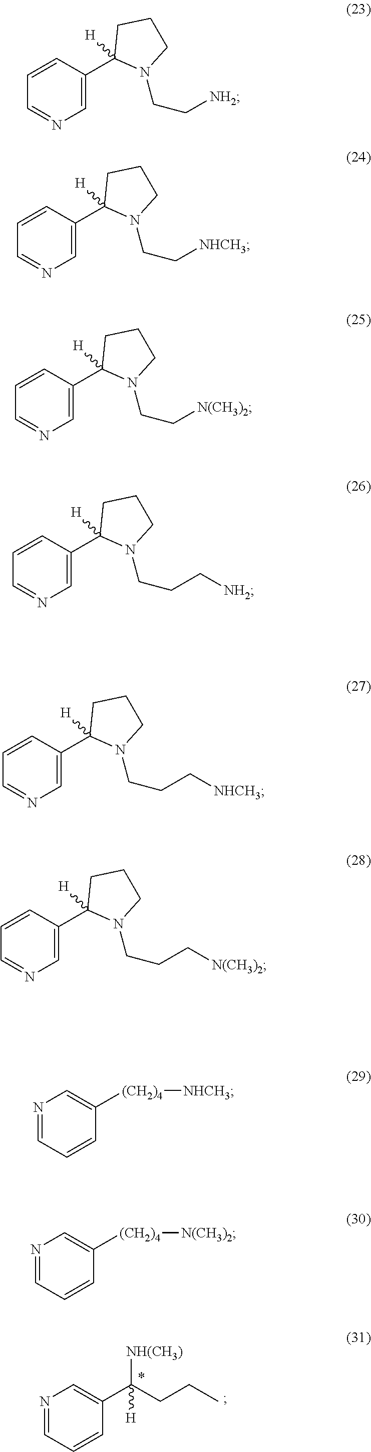

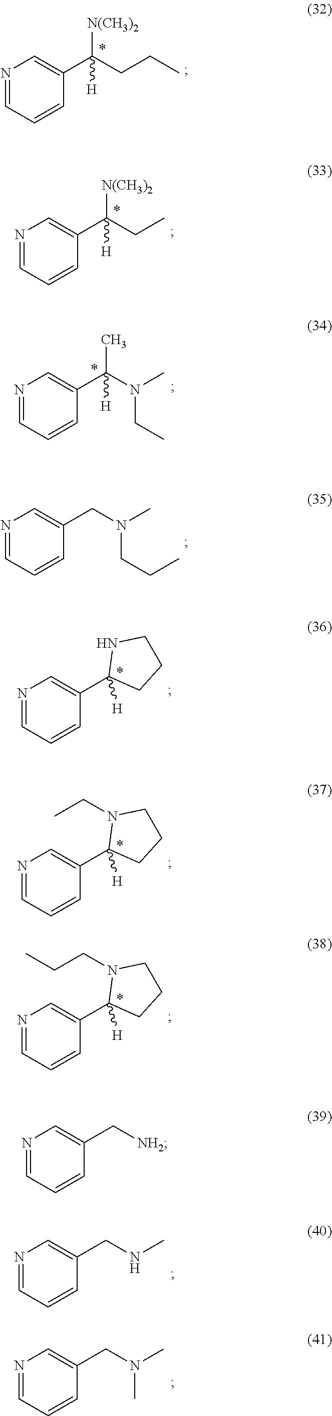

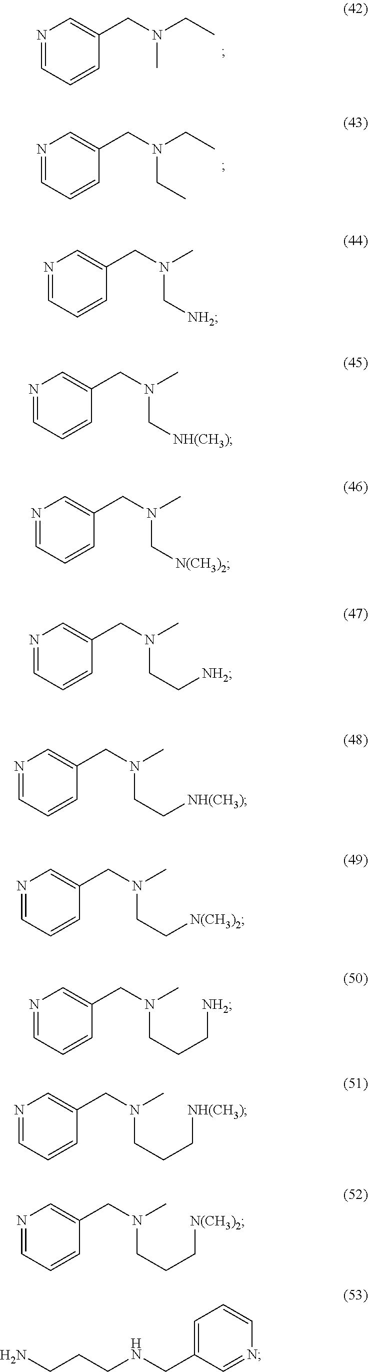

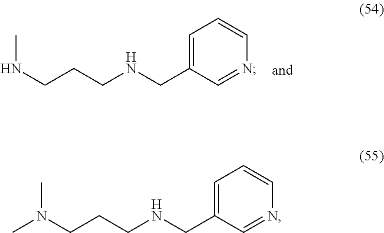

or a salt, or a mixed salt thereof.

In various embodiments, a THR product herein comprises at least one substituted pyridine compound, or salt, or mixed salt thereof, according to Formula (IAA):

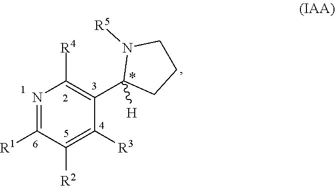

wherein R¹, R², R³, R⁴, and R⁵ are as previously defined, with the proviso that if R⁵ is CH₃ in Formula (IAA), then R¹, R², R³, and R⁴ cannot all be H. This proviso language expressly excludes both (R) and (S) enantiomers of nicotine from consideration in the compositions according to the present disclosure, as for genus structure (I) and its subgenus structures.

As noted from the delineated selections for a substituted pyridine compound, many have a chiral center that can be either (R) or (S). In most examples, the (S) enantiomer is preferred, and the (R) enantiomer might be purposely excluded due to possible toxicity or undesired physiological effects, or at least for its inability to provide any cognizable benefit. In various examples, a substituted pyridine compound in accordance with Formula (I) having a chiral center is a racemic mixture of (R) and (S) at the chiral center (*), such as obtained by a nonchiral synthesis. The (R) or (S) enantiomer may be obtained by a chiral synthesis or by separation of enantiomers from a racemic mixture.

In various embodiments, a substituted pyridine compound for use herein has a structure of Formula (IAA),

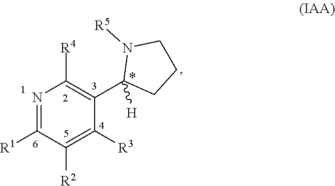

and independent of R⁵, at least one of R¹, R², R³, and R⁴ is methyl, and the remaining R¹, R², R³, and R⁴ substituents that are not methyl are H.

In various embodiments of Formula (IAA), R⁵ is CH₃ and at least one of R¹, R², R³, and R⁴ is not H.

In various embodiments of Formula (IAA), R¹, R², R³, R⁴ and R⁵ are each H.

In various embodiments of Formula (IAA), R⁵ is —(CH₂)n-NR¹⁵R¹⁶, wherein n is an integer from 1 to 10, and wherein R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered optionally substituted heterocyclic ring, including for example, aziridine, azetidine, pyrrolidine, piperidine, 1,4-piperazine, and morpholine. In various embodiments, and independent of R¹, R², R³, and R⁴, R⁵ is selected from —(CH₂)—NH₂; —(CH₂)₂—NH₂; —(CH₂)₃—NH₂; —(CH₂)₄—NH₂; —(CH₂)₅—NH₂; —(CH₂)₆—NH₂; —(CH₂)₇—NH₂; —(CH₂)₈—NH₂; —(CH₂)₉—NH₂; —(CH₂)₁₀—NH₂; —(CH₂)—N(CH₃)₂; —(CH₂)₂—N(CH₃)₂; —(CH₂)₃—N(CH₃)₂; —(CH₂)₄—N(CH₃)₂; —(CH₂)₅—N(CH₃)₂; —(CH₂)₆—N(CH₃)₂; —(CH₂)₇—N(CH₃)₂; —(CH₂)₈—N(CH₃)₂; —(CH₂)₉—N(CH₃)₂; and —(CH₂)₁₀—N(CH₃)₂.

In various embodiments, R⁵ is H, and at least one of R¹, R², R³, and R⁴ is —(CH₂)n-NR⁶R⁷, wherein n is an integer from 1 to 10, and wherein R⁶ and R⁷ are independently selected from H, alkyl, substituted alkyl, cycloalkyl, substituted cycloalkyl, alkenyl and substituted alkenyl, or R⁶ and R⁷ together with the N atom to which they are bonded form a 3-8 membered optionally substituted heterocyclic ring.

In various embodiments, a substituted pyridine compound for use herein has a structure of Formula (IAA),

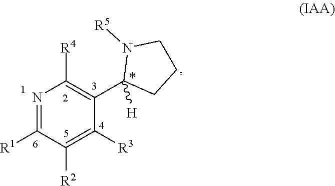

wherein R⁵ is CH₃, and at least one of R¹, R², R³, and R⁴ is —(CH₂)n-NR¹⁵R¹⁶, wherein n is an integer from 1 to 10, and wherein R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered optionally substituted heterocyclic ring.

In various embodiments of Formula (IAA), R⁵ is CH₃, and at least one of R¹, R², R³, and R⁴ is —(CH₂)n-N(CH₃)₂, wherein n is an integer from 1 to 10.

In various embodiments, substituted pyridine compounds of Formula (IAA) for use in THR products herein include, but are not limited to, (R) or (S)-3-(pyrrolidin-2-yl)pyridine, (R) or (S)-2-methyl-3-(1-methylpyrrolidin-2-yl)pyridine, (R) or (S)-4-methyl-3-(1-methylpyrrolidin-2-yl)pyridine, (R) or (S)-3-methyl-5-(1-methylpyrrolidin-2-yl)pyridine, (R) or (S)-2-methyl-5-(1-methylpyrrolidin-2-yl)pyridine, (R) or (S)-2,6-dimethyl-3-(1-methylpyrrolidin-2-yl)pyridine, (R) or (S)-2,4-dimethyl-5-(1-methylpyrrolidin-2-yl)pyridine, (R) or (S)-2,3-dimethyl-5-(1-methylpyrrolidin-2-yl)pyridine, (R) or (S)-2,4-dimethyl-3-(1-methylpyrrolidin-2-yl)pyridine, (R) or (S)-2,5-dimethyl-3-(1-methylpyrrolidin-2-yl)pyridine, (R) or (S)-3,4-dimethyl-5-(1-methylpyrrolidin-2-yl)pyridine, (R) or (S)-1-(2-aminoethyl)-2-(3-pyridyl)pyrrolidine, (R) or (S)-1-(3-aminopropyl)-2-(3-pyridyl)pyrrolidine, (R) or (S)-1-(4-aminobutyl)-2-(3-pyridyl)pyrrolidine, (R) or (S)-1-(5-aminopentyl)-2-(3-pyridyl)pyrrolidine, (R) or (S)-1-(2-dimethylaminoethyl)-2-(3-pyridyl)pyrrolidine, (R) or (S)-1-(3-dimethylaminopropyl)-2-(3-pyridyl)pyrrolidine, (R) or (S)-1-(4-dimethylaminobutyl)-2-(3-pyridyl)pyrrolidine, (R) or (S)-3-(2-(6-methylpyridin-3-yl)pyrrolidin-1-yl)propanenitrile, (R) or (S)-4-(2-(6-methylpyridin-3-yl)pyrrolidin-1-yl)butanenitrile, (R) or (S)-1-(3-aminopropyl)-2-(6-methyl-3-pyridyl)pyrrolidine, (R) or (S)-1-[3-(N,N-dimethylamino)propyl]-2-(6-methyl-3-pyridyl)pyrrolidine, (R) or (S)-1-(N,N-diethyl-3-aminopropyl-2-(3-pyridyl)pyrrolidine, (R) or (S)-3-(1-(2-(pyrrolidin-1-yl)ethyl)pyrrolidin-2-yl)pyridine, (R) or (S)-3-(1-(2-(piperidin-1-yl)ethyl)pyrrolidin-2-yl)pyridine, (R) or (S)-4-(2-(2-(pyridin-3-yl)pyrrolidin-1-yl)ethyl)morpholine, (R) or (S)-3-(1-(3-(pyrrolidin-1-yl)propyl)pyrrolidin-2-yl)pyridine, (R) or (S)-3-(1-(3-(piperidin-1-yl)propyl)pyrrolidin-2-yl)pyridine, and (R) or (S)-4-(3-(2-(pyridin-3-yl)pyrrolidin-1-yl)propyl)morpholine.

In each of the alkaloid examples herein having a chiral center, the (S) enantiomer is likely active as a nicotinic acetylcholine receptor ligand and is preferred, whereas the (R) enantiomer is likely physiologically inert or possibly even metabolically toxic. Thus, in preferred embodiments, a racemic mixture of any of these alkaloids is not used in any of the THR products herein.

In various embodiments of Formula (IAA), R¹, R², R³, R⁴ and R⁵ are independently selected from H, methyl, ethyl, n-propyl, iso-propyl, tert-butyl, cyclopropyl, cyclobutyl, cyclopentyl and cyclohexyl, with the proviso that if R⁵ is methyl, then R¹, R², R³, and R⁴ cannot all be H.

In preferred examples of Formula (IAA), R¹ and R⁵ are both methyl, R², R³, and R⁴ are each H, and the alkaloid is in the (S)-configuration.

### Methods of Use

### 1. Smoking Cessation Through THR Products Used Singly or in Combination

As mentioned herein above, one goal of the present disclosure is to provide THR products to a current tobacco smoker desirous of, and engaged in, a smoking cessation program. In certain examples, THR products are used in combination so as to provide the individual with a sense of smoking and to alleviate what is perceived to be a physical pain from not smoking. In certain examples, an individual on a cessation program will be prescribed an oral THR product and a topical cream THR product for joints and muscles that may be perceived as “aching.”

In various embodiments, a method of smoking cessation comprises administering to an individual desirous of cessation one or more THR products over a time period determined for cessation, each THR product comprising at least one substituted pyridine compound, or salt or mixed salt thereof, having Formula (I):

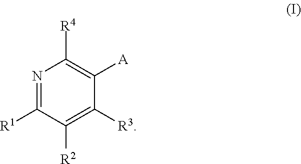

In various embodiments, the time period determined for cessation may be days, weeks, months or years in length, preferably several months to a year in length.

In various embodiments, the time period determined for cessation is part of a cessation program that further includes dispensation of a series of THR products over the time period determined for cessation.

In various embodiments, the time period determined for cessation is divided into consecutive time periods of shorter duration, wherein during each one of the shorter time periods the individual desirous of cessation receives and uses a different THR products, optionally in combination, in place of tobacco smoking. At the end of the program, the individual desirous of cessation will be using only THR products as presented herein, which by definition are devoid of

In various embodiments, an individual enrolled in a smoking cessation program will check in with their program manager on a regular basis, such as weekly, for personal consultation and to receive different, modified, or other THR products, such as different combinations of THR products. At these regular check-ins, an assessment can be made as to the individual's compliance with the program, and adjustments made accordingly. Some changes to a program may be to keep an individual on a certain w/v level of substituted pyridine compound for a longer period of time than a week, or to change or adjust flavoring agents.

One important aspect of the present disclosure is use of a combination of THR products for smoking cessation. As mentioned, withdrawal from cigarette smoking can induce, perhaps physiologically or psychologically, physical pain. Whether or not this pain is real, certain THR products herein are designed to treat pain, particularly the topical creams and pain relief patches. These types of THR products can be prescribed for smoking cessation in combination with one or more products designed to orally deliver on of the substituted pyridine compounds, such as a chewing gum or sublingual tablets.

In the detailed description, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected, coupled or the like may include permanent (e.g., integral), removable, temporary, partial, full, and/or any other possible attachment option. Any of the components may be coupled to each other via friction, snap, sleeves, brackets, clips or other means now known in the art or hereinafter developed. Additionally, any reference to without contact (or similar phrases) may also include reduced contact or minimal contact.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

All structural, chemical, and functional equivalents to the elements of the above-described various embodiments that are known to those of ordinary skill in the art are expressly incorporated herein by reference and are intended to be encompassed by the present claims. Moreover, it is not necessary for an apparatus or component of an apparatus, or method in using an apparatus to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

What is claimed is:

1. A tobacco harm reduction (THR) product comprising:
at least one substituted pyridine compound of Formula (I); 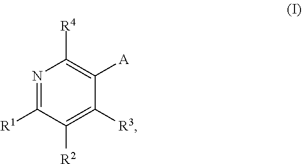
or a salt, or mixed salt thereof, wherein:
A is selected from: 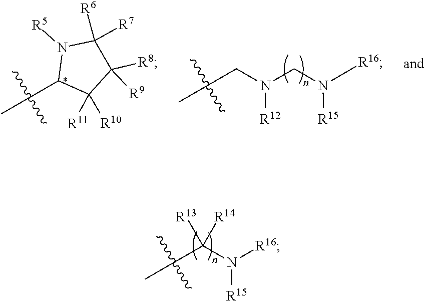
R¹, R², R³, R⁴ and R⁵ are independently selected from H, alkyl, substituted alkyl, cycloalkyl, substituted cycloalkyl, alkenyl, substituted alkenyl, and 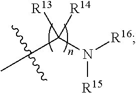
R⁶ and R⁷ are independently selected from H, alkyl, substituted alkyl, cycloalkyl, substituted cycloalkyl, alkenyl and substituted alkenyl, or R⁶ and R⁷ together are ═O;
R⁸, R⁹, R¹⁰, R¹¹, R¹², R¹³, and R¹⁴ are independently selected from H, alkyl, substituted alkyl, cycloalkyl, substituted cycloalkyl, alkenyl, and substituted alkenyl;
n is an integer from 1 to 10; and
R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered substituted heterocyclic ring,
with the proviso that if A is 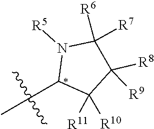
and R⁵ is methyl, then R¹, R², R³, R⁴, R⁶, R⁷, R⁸, R⁹, R¹⁰, and R¹¹ cannot all be H; and
wherein the THR product is entirely devoid of (R) or (S) nicotine.

2. The THR product of claim 1, further comprising at least one excipient, at least one organic acid, at least one chemosensory irritant, at least one flavoring agent, a substrate and combinations thereof.

3. The THR product of claim 2, wherein the excipient is selected from a binder, a diluent, a syrup, a sugar, a sweetener, a solubilizer, a solvent, a polyol, an emollient, a humectant, an oil, a polymer, a surfactant, a carrier, a stabilizer, a preservative, and mixtures thereof.

4. The THR product of claim 2, wherein the excipient or the substrate contribute to a physical form or a function of the THR product.

5. The THR product of claim 2, wherein the substrate is selected from a nonwoven, a latex, rubber or fabric swatch, a wood splint, or dental string or tape.

6. The THR product of claim 1, comprising (a) a transdermal patch, (b) a dissolving film, (c) a lozenge, (d) a chewing gum, (e) an oral pouch, (f) impregnated toothpicks, (g) impregnated or coated dental floss, (h) a mouthwash, (i) a gummy candy, (j) sublingual disintegrating tablets, (k) a topical/transdermal cream, (l) an oral spray, (m) inhaler, (n) nasal spray, (o) alkaloid dispensing pouch, (p) e-cigarette, (q) electronic nicotine delivery system or comparable nicotine replacement product.

7. The THR product of claim 1, wherein a molar ratio of moles substituted pyridine compound of Formula (I) to moles total organic acid is from about 25:1 to about 75:1.

8. The THR product of claim 1, wherein the at least one substituted pyridine compound is selected from (R) or (S)-3-(pyrrolidin-2-yl)pyridine, (R) or (S)-2-methyl-3-(1-methylpyrrolidin-2-yl)pyridine, (R) or (S)-4-methyl-3-(1-methylpyrrolidin-2-yl)pyridine, (R) or (S)-3-methyl-5-(1-methylpyrrolidin-2-yl)pyridine, (R) or (S)-2-methyl-5-(1-methylpyrrolidin-2-yl)pyridine, (R) or (S)-2,6-dimethyl-3-(1-methylpyrrolidin-2-yl)pyridine, (R) or (S)-2,4-dimethyl-5-(1-methylpyrrolidin-2-yl)pyridine, (R) or (S)-2,3-dimethyl-5-(1-methylpyrrolidin-2-yl)pyridine, (R) or (S)-2,4-dimethyl-3-(1-methylpyrrolidin-2-yl)pyridine, (R) or (S)-2,5-dimethyl-3-(1-methylpyrrolidin-2-yl)pyridine, (R) or (S)-3,4-dimethyl-5-(1-methylpyrrolidin-2-yl)pyridine, 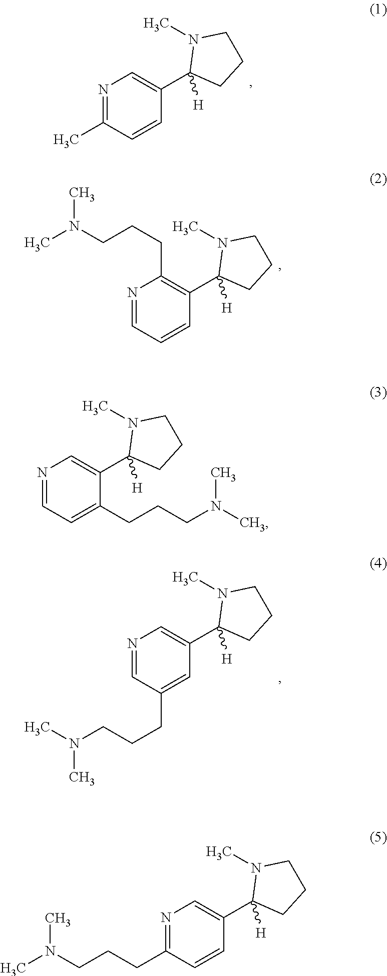 [SMILES: Cc1ccc(C2CCCN2C)cn1 ; CN(C)CCCc1ncccc1C1CCCN1C ; CN(C)CCCc1ccncc1C1CCCN1C ; CN(C)CCCc1cncc(C2CCCN2C)c1 ; CN(C)CCCc1ccc(C2CCCN2C)cn1] 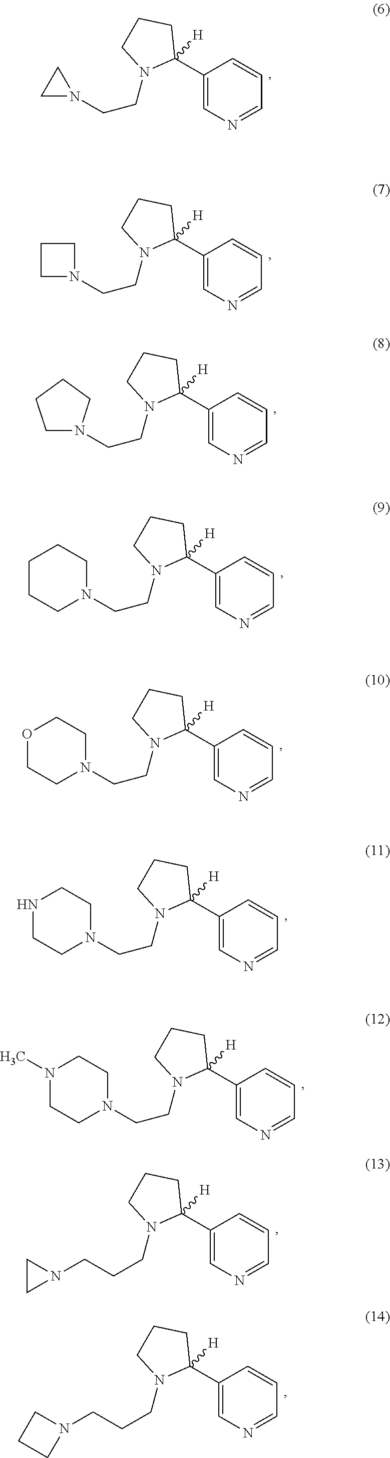 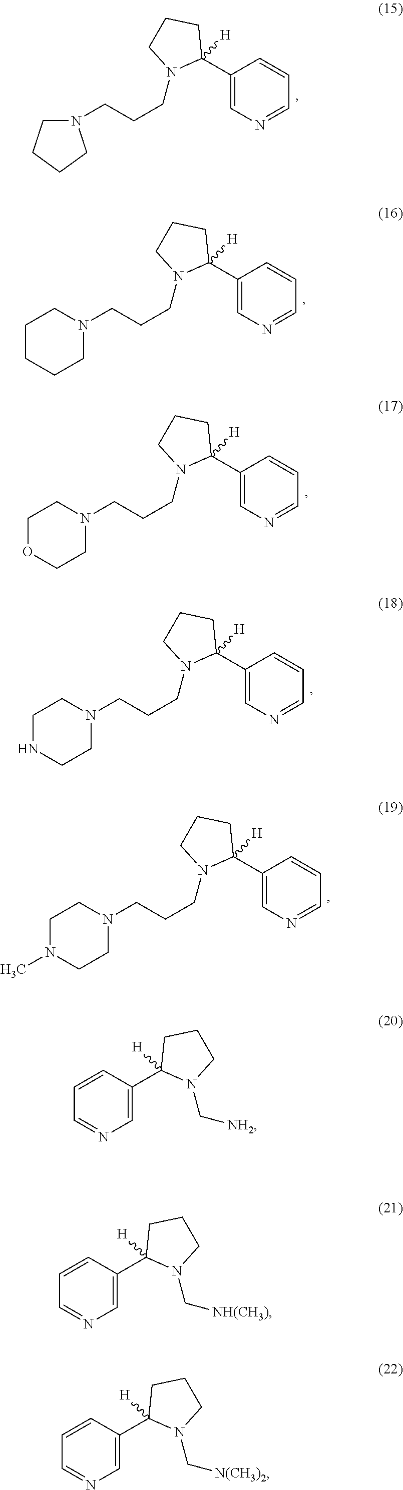 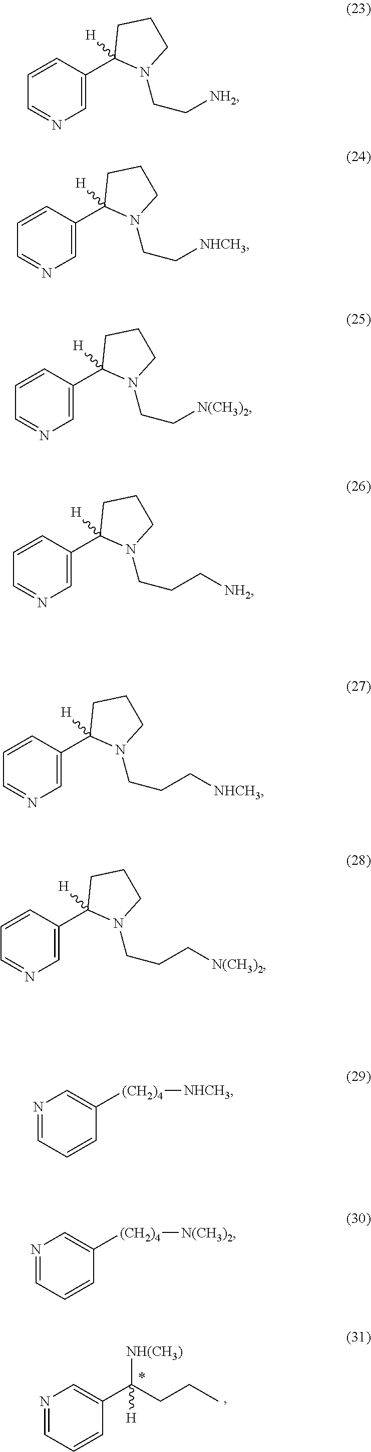 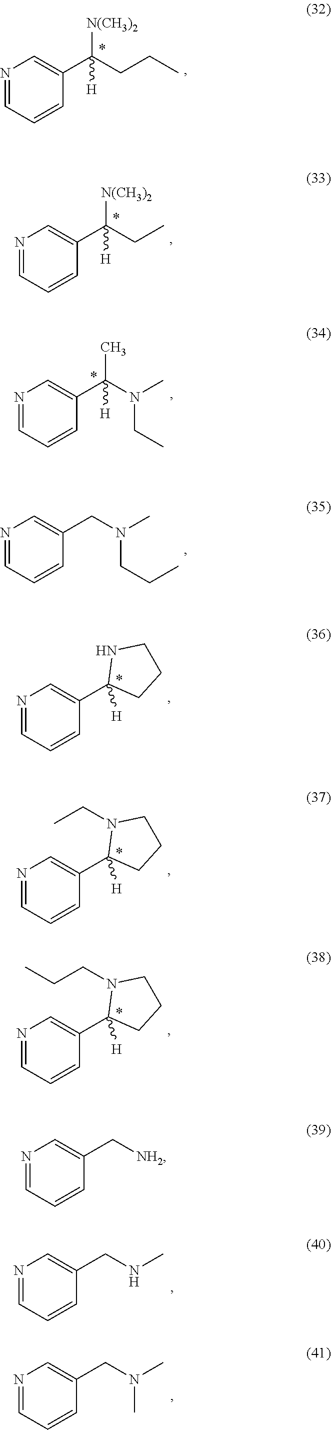 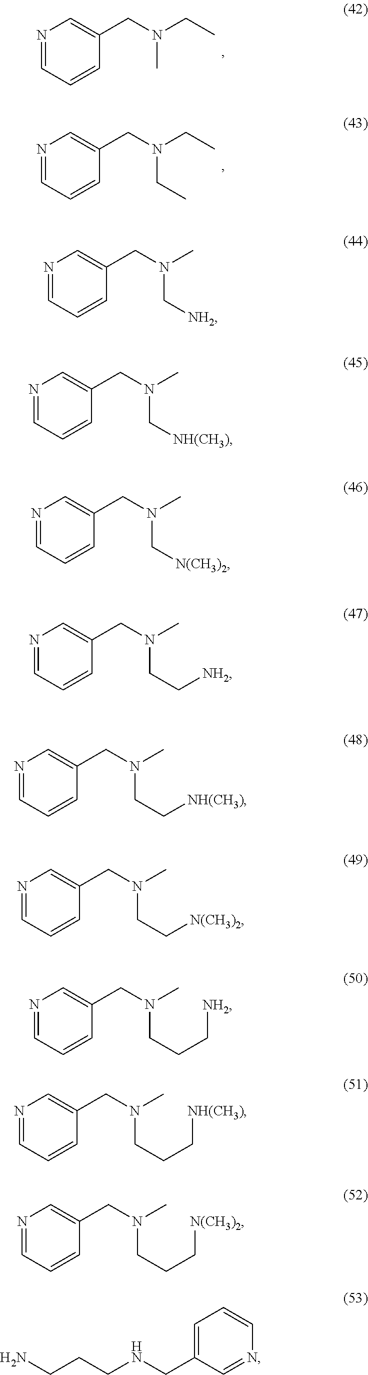 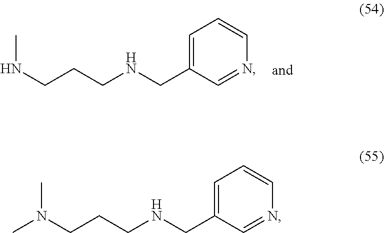
or a salt, or a mixed salt thereof.

9. The THR product of claim 8, wherein the molar ratio of moles of (S)-2-methyl-5-(1-methylpyrrolidin-2-yl)pyridine to total moles of organic acid is about 50:1.

10. The THR product of claim 1, wherein the at least one substituted pyridine compound of Formula (I), or salt thereof, or mixed salt thereof, consists of a mixture of (S)-2-methyl-3-(1-methylpyrrolidin-2-yl)pyridine free base and (2S)-1-methyl-2-(6-methylpyridin-3-yl)pyrrolidin-1-ium citrate/acetate mixed salts.

11. The THR product of claim 1, wherein:
A is

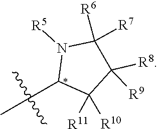

12. The THR product of claim 1, wherein:
A is

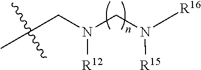

13. The THR product of claim 1, wherein:
A is

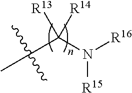

14. A tobacco harm reduction (THR) product comprising:
from about 1 wt. % to about 80 wt. % of a substituted pyridine compound of Formula (I); 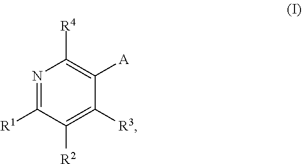
or a salt, or mixed salt thereof, wherein:
A is selected from: 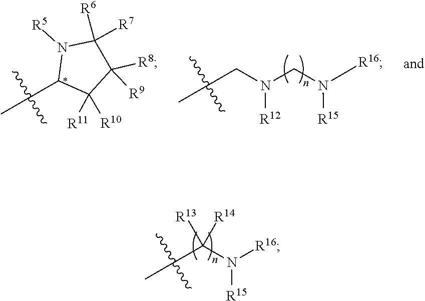
R¹, R², R³, R⁴ and R⁵ are independently selected from H, alkyl, substituted alkyl, cycloalkyl, substituted cycloalkyl, alkenyl, substituted alkenyl, and 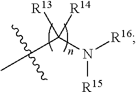
R⁶ and R⁷ are independently selected from H, alkyl, substituted alkyl, cycloalkyl, substituted cycloalkyl, alkenyl and substituted alkenyl, or R⁶ and R⁷ together are ═O;
R⁸, R⁹, R¹⁰, R¹¹, R¹², R¹³, and R¹⁴ are independently selected from H, alkyl, substituted alkyl, cycloalkyl, substituted cycloalkyl, alkenyl, and substituted alkenyl;
n is an integer from 1 to 10; and
R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered substituted heterocyclic ring,
with the proviso that if A is 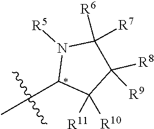
and R⁵ is methyl, then R¹, R², R³, R⁴, R⁶, R⁷, R⁸, R⁹, R¹⁰, and R¹¹ cannot all be H; and
wherein the THR product is entirely devoid of (R) or (S) nicotine.

15. A method for promoting smoking cessation in an individual desirous of cessation, the method comprising:
administering to the individual, over a time period determined for cessation, a series of alkaloid compositions for the individual to use, each alkaloid composition in the series comprising at least one substituted pyridine compound according to Formula (I): 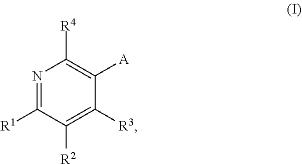
or a salt, or mixed salt thereof, wherein:
A is selected from: 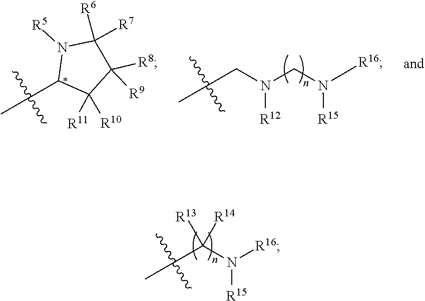
R¹, R², R³, R⁴ and R⁵ are independently selected from H, alkyl, substituted alkyl, cycloalkyl, substituted cycloalkyl, alkenyl, substituted alkenyl, and 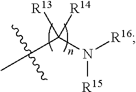
R⁶ and R⁷ are independently selected from H, alkyl, substituted alkyl, cycloalkyl, substituted cycloalkyl, alkenyl and substituted alkenyl, or R⁶ and R⁷ together are ═O;
R⁸, R⁹, R¹⁰, R¹¹, R¹², R¹³, and R¹⁴ are independently selected from H, alkyl, substituted alkyl, cycloalkyl, substituted cycloalkyl, alkenyl, and substituted alkenyl;
n is an integer from 1 to 10; and
R¹⁵ and R¹⁶ are independently selected from H, alkyl, substituted alkyl, cycloalkyl, substituted cycloalkyl, alkenyl and substituted alkenyl, or R¹⁵ and R¹⁶ together with the N atom to which they are bonded form a 3-8 membered substituted heterocyclic ring,
with the proviso that if A is 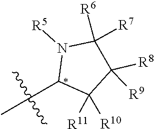
and R⁵ is methyl, then R¹, R², R³, R⁴, R⁶, R⁷, R⁸, R⁹, R¹⁰, and R¹¹ cannot all be H; and
wherein the THR product is entirely devoid of (R) or (S) nicotine.

16. The method of claim 15, wherein the individual inhales by vaping each composition in the series beginning at a start of the time period determined for cessation with the alkaloid composition having the highest level (w/v) of said substituted pyridine compound of Formula (I), or salt, or mixed salt thereof, and ending at an end of the time period determined for cessation with the composition having the lowest level (w/v) of said substituted pyridine compound of Formula (I), or salt, or mixed salt thereof, such that at commencement of the time period determined for cessation the individual ceases smoking.

17. The method of claim 15, wherein the individual (a) applies a transdermal patch, (b) applies a dissolving film, (c) consumes a lozenge, (d) consumes a chewing gum, (e) consumes an oral pouch, (f) lick impregnated toothpicks, (g) use impregnated or coated dental floss, (h) consume a mouthwash, (i) consume a gummy candy, (j) consume sublingual disintegrating tablets, or (k) apply a topical/transdermal cream.

18. The method of claim 15, wherein the alkaloid composition in the series of vaporizable alkaloid compositions having the highest level (w/v) of said substituted pyridine compound of Formula (I), or salt, or mixed salt thereof comprises about 100 mg/mL of said substituted pyridine compound of Formula (I), or salt, or mixed salt thereof.

19. The method of claim 15, wherein the alkaloid composition in the series of vaporizable alkaloid compositions having the lowest level (w/v) of substituted pyridine compound of Formula (I), or salt, or mixed salt thereof comprises no more than about 1 mg/mL of said substituted pyridine compound of Formula (I), or salt, or mixed salt thereof.

20. The method of claim 15, wherein the time period determined for cessation is part of a smoking cessation program that further includes dispensation of the series of vaporizable alkaloid compositions to the individual desirous of cessation over the time period determined for cessation.
